(19) United States
(12) Patent Application Publication
(10) Pub. No.: US 2026/0358282 A1
(43) Pub. Date: Nov. 12, 2026

(54) SYSTEMS AND METHODS FOR QUALITY VERIFICATION FOR A MIXTURE

(71) Applicant: 3M INNOVATIVE PROPERTIES COMPANY, St. Paul, MN (US)

(72) Inventors: Benjamin J. Muenstermann, Duisburg (DE); Christian Weinmann, Alsdorf (DE); Christopher J. Shaffer, St. Paul, MN (US); David M. Rudek, Duesseldorf (DE); Joerg Hahn, Bad Neuenahr-Ahrweiler (DE); Knut Schumacher, Neuss (DE); Michael H. Stalder, Uedem (DE); Robert J. Bialluch, Willich (DE); Robert J. Franzwa, Lake Elmo, MN (US); Ryan D. Erickson, Roseville, MN (US); Ryan P. Marrinan, Lino Lakes, MN (US); Sai Bhargav Jampu, Neuss (DE); Simon Plugge, Duesseldorf (DE); Waleri Wischnepolski, Neuss (DE)

(21) Appl. No.: 19/135,105

(22) PCT Filed: Dec. 8, 2023
(86) PCT No.: PCT/IB2023/062403

§ 371 Date: Jun. 3, 2025

Related U.S. Application Data
(60) Provisional application No. 63/386,715, filed on Dec. 9, 2022.
(60) Provisional application No. 63/486,631, filed on Feb. 23, 2023.
(60) Provisional application No. 63/507,662, filed on Jun. 12, 2023.

(51) Int. Cl. G01N 27/07; G01K 13/02; G01N 27/10; G01N 27/20; G01N 27/22; G01N 27/24; G01N 33/32

(52) U.S. Cl. CPC G01N 27/07; G01K 13/026; G01N 27/10; G01N 27/20; G01N 27/226; G01N 27/24; G01N 33/32

(57) ABSTRACT

An electrical property sensor is presented that includes a laminate structure including an insulating layer, a conductive layer and a conductive trace, the laminate structure having a first face separated from a second face by a thickness, the first face having a length and a width. The sensor also includes a first and second aperture, each of the first and second apertures extending from the first face of the laminate structure to the second face of the printed circuit board, the first and second aperture each include a receiving electrode and a transmitting electrode. When a fluid flows through the first aperture and an electric field is generated at the transmitting electrode, a sensor signal indicative of an electrical property is measured at the receiving electrode.

### BACKGROUND

Many products require mixtures in manufacture—e.g. paint for commercial or industrial use, adhesives, resins, etc. Many mixtures include different materials that, over time, may settle or separate. It may not be easily recognized by a user of the mixture that the composition is no longer consistent.

### SUMMARY OF THE DISCLOSURE

An electrical property sensor is presented that includes a laminate structure including an insulating layer, a conductive layer and a conductive trace, the laminate structure having a first face separated from a second face by a thickness, the first face having a length and a width. The sensor also includes a first and second aperture, each of the first and second apertures extending from the first face of the laminate structure to the second face of the printed circuit board, the first and second aperture each include a receiving electrode and a transmitting electrode. When a fluid flows through the first aperture and an electric field is generated at the transmitting electrode, a sensor signal indicative of an electrical property is measured at the receiving electrode.

Systems and methods including such sensors allow for direct contact between the sensor and a fluid flowing through a dispenser as sensors herein are cost effective to manufacture and can be discarded after use. Systems and methods herein also allow for multiple sensor signals to be gathered across a fluid flow, providing real-time information about materials going into, and out of, a mixing area. Systems and methods herein also allow for bubble detection and removal. Systems and methods herein allow for dispensing systems and their operators to change operational parameters during an operation to address issues as they are occurring, or potentially before the occur, such that less material is wasted and more accurate dispensing is possible.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF FIGURES

FIGS. 1A-1C illustrate systems for dispensing an atomized fluid that may benefit from systems and methods herein.

FIG. 2 illustrates an exploded view of a spray gun, in which embodiments described herein may be implemented.

FIGS. 3A-3B illustrate material measurement flow sensors in accordance with embodiments herein.

FIGS. 4A-4B illustrate spray guns in accordance with embodiments herein.

FIGS. 5A & 5B illustrate material measurement flow sensors as used in accordance with embodiments herein.

FIGS. 6A-6B, and 7A-D illustrate example implementations of dispensing systems with sensor systems installed in accordance with embodiments herein.

FIGS. 8A-8C illustrate dispersion states that may be experienced using sensor systems and methods described herein.

FIG. 9 illustrates a stir stick configured to provide in-situ conductivity measurements for a mixture.

FIG. 10 illustrates an elongated sensor in accordance with embodiments herein.

FIGS. 11A-11D illustrates a sensor with electrodes in a series configuration in accordance with embodiments herein.

FIG. 12 illustrates another embodiment of a system in which embodiments herein may be useful.

FIGS. 13A-13C illustrate a sensor configuration for bubble detection in accordance with embodiments herein.

FIG. 14 illustrates a method for detecting and correcting quality concerns in a mixture in accordance with embodiments herein.

FIG. 15 illustrates a quality control system, in accordance with embodiments herein.

FIGS. 16A-16B illustrate sensor configurations in accordance with embodiments herein.

FIG. 17 illustrates a method of quality controlling a material dispensing system in accordance with embodiments herein.

FIGS. 18A-18C illustrates conductivity measurement system in example network architectures.

FIGS. 19-21 illustrates example computing devices that can be used in embodiments herein.

FIGS. 22-24 illustrate results described in the Examples.

### DETAILED DESCRIPTION OF ILLUSTRATIVE EMBODIMENTS

The present disclosure relates to systems and methods that include sensors that determine properties of fluids in-situ. The disclosure also relates to data sets received by such sensors and methods of using said data for analyzing said fluid properties. Using systems and methods described herein, it may be possible to adjust use conditions of a mixture (e.g. change pressure, temperature, mix ratio, etc.) or to improve composition consistence (e.g. re-mix, off-gas, etc.) before, or during, an operation.

Many industrial processes use mixtures such as liquid adhesives, liquid food ingredients, liquid coolants, or liquid reaction products, to name a few examples. Certain properties of such liquids vary over time: a dispersion or emulsion may separate, an oil may become less viscous as temperature rises, a coolant may age and have a lower heat capacity than initially. Performance of the product as used may suffer. For example, paint may have a soft cure (or no cure at all), may be brittle, crack, experience delamination or poor adhesion. If a paint mixture is not consistent before application, corrective action may take considerable time —energy-intensive sanding and surface preparation may be required before the painting operation is attempted a second time. Troubleshooting these issues require detailed chemical knowledge, time and elimination of other causes. For many operations, troubleshooting costs time that cannot be spared.

Co-pending international application IB2021/056362, filed on Jul. 14, 2021, discloses a property sensor for determining a property value of a liquid that includes two PCB boards that define a channel through which the liquid flows. While this allows for direct contact between the sensor and the fluid, there exists a need for cost-effective sensors that can provide more contextual information about material mixing. Embodiments herein provide systems and methods for effectively and accurately measuring material information for mixture quality control.

Described herein are sensors and sensor systems that are used to measure electrical properties of fluids. Broadly sensors herein function by a transmitting electrode, using a provided current or voltage, creates an electrical field. As a fluid flows between the transmitting electrode and a receiving electrode, it conducts a current to the receiving electrode. The term “sensor” as used herein may refer both to the physical sensor that provides a sensor signal indicative of conducted current, as well as to a “sensor system” that includes a processor that calculates an electrical property of the fluid based on the sensor signal.

The term “electrical property” is intended to broadly refer to any electrical property of a fluid that can be derived based on impedance measurements of a sensor. Used herein, for ease of understanding the embodiments, are the example of impedance measurements. However, it is expressly contemplated that other electrical properties may be calculated and relevant to embodiments herein. For example, conductivity measurements or dielectric constants may also be determined from impedance measurements. Either conductivity or dielectric constant may be relevant, as illustrated herein, for determining relevant functionality of a dispensing system or quality of fluids flowing therein.

As used herein, the term “real-time” refers to data is processed within milliseconds so that it is available virtually immediately as feedback. While some delay due to processing are inevitable, “real-time” is intended to cover systems and methods where data can be collected or entered and a user can then interact with it without noticeable delay. E.g. a user may make a data entry into a system, and the data entry is then substantially immediately available for viewing or editing.

As described herein, sensors are described as measuring electrical properties of “fluids.” The term “fluid” is intended to be interpreted broadly and is intended to cover liquids with low viscosities, liquids with high viscosities, semi-solid materials, suspensions, melted materials, or other flowable materials.

Electrical parameters, as used herein, may be detected by an electrode pair. Fluid may flow between or past the electrode pair. A transmitting electrode may generate an electric field when a voltage or a current is applied, while a receiving electrode receives a current or voltage. The sensed electrical parameter may be a conductivity, relative permittivity or an impedance. The terms relative permittivity and dielectric constant are used herein interchangeably.

Sensors are described herein as having one or more “apertures” within a “printed circuit board.” These terms are intended to be interpreted broadly. For example, an aperture may fully extend through a thickness of a sensor along part of, or the entirety of its length. Apertures may have beveling along part or all of a perimeter. An aperture may be elongated, such as a slot, or may be shaped, such as a circular or ovular hole. An aperture may have one or more corners or edges, or may have curvature along part or all of its perimeter. As used herein, a “printed circuit board” refers to a laminated sandwich structure of conductive and insulating layers. Printed circuit boards (PCBs) herein may include any number of terminals and conductors that allow for voltage to be applied to a transmitting electrode and for current to be transmitted from a receiving electrode. Alternatively, PCBs may also be constructed to allow for a current to be applied and voltage transmitted. PCBs may be manufactured using traditional PCB manufacturing technology or additive manufacturing technology. As used herein, PCB is intended to cover any number of layers, with or without an edge connector. Any suitable conductive metal may be used to form conductive layers. Any suitable insulating material may be used to form insulating layers.

Property sensors as described herein may be used to sense properties of a fluid resulting from a mixing process. They may also be used to sense properties of input fluids for a mixing process or for an industrial manufacturing process. Advantageously, separate property sensors for respective input fluids are placed just in front of the mixer. Data from these property sensors measuring the input fluids can be processed along with data from a property sensor measuring the mixed fluid, e.g. in an integrated materials property monitoring system. Where, for example, a fluid composition is mixed from three input fluids, a property of each of the three fluids before mixing can be determined using three property sensors at the respective outlets of the three containers containing the three input fluids. This may help in quality control and reduce waste that might otherwise occur due to one of the input fluids being outside a specification for the property.

Sensors described herein may determine various properties of a fluid, like, for example, mixing ratio of a two-component adhesive or curing status of a curable composition or ageing status.

The term “curing” as used herein is intended to broadly cover a changing of a material from a first state to a second state. For example, some liquids cure into solids. Some mixtures may experience crosslinking. Some mixtures may experience pre-polymerization. Some mixtures may experience conversion. The number of properties which were varied previously to establish the set of calibration data representing calibration impedance responses measured previously at the different property values determines the number of properties that can later be determined by the property sensor. The pre-stored set of calibration data representing calibration impedance responses measured previously at the one or more sensing frequencies and at different property values of a property of the fluid forms, or represents, a multi-dimensional data field which is specific for the fluid. This data field allows the property value deriver to determine, from a response impedance actually measured, a value of the property of the fluid.

A fluid has many properties: for example, viscosity, density, color, content of volatile components, water content, chemical composition, boiling point, but also ageing status, curing status in case of fluid curable compositions, or mixing ratio in case of the fluid being a mixture, to name only some.

Further, certain properties of certain fluids, however, vary with time and/or with other parameters such that the response impedance in a property sensor described herein varies with time and/or with the other parameters, too. Values of these properties may be derived via sensors and systems described herein. Additionally, variation with time includes variation of the property between different production lots of the fluid. The property sensor described herein can thus be used to detect differences in a certain property (e.g. chemical composition) of a suitable fluid between a later production lot and an earlier production lot of the fluid.

As used herein, “fluids” may be part of a mixture. For example, one fluid may mix with another fluid, may receive or have particles dissolved therein, or may have materials suspended therein. For example, a resin may include glass particulates in one or both components.

The term “property” of the fluid, according to the present disclosure, is not particularly limited. For example, as described in embodiments herein, one property of interest is a mixing ratio of two or more components of the fluid. In certain of these embodiments, the fluid is a two-component adhesive, and a property of the fluid is a mixing ratio of the components. In other embodiments, a property of interest is a curing degree or a curing status. In certain of these embodiments the fluid is a curable composition, and a property of the fluid is the degree of curing of the composition.

In other embodiments, a property of interest is an ageing degree or an ageing status. In certain of these embodiments the fluid is an ageing fluid, i.e. a fluid in which certain characteristics change over time once the ageing fluid has been created. The property sensor may determine a change in the response impedance of the ageing fluid after some ageing, compared to response impedances of an identical fluid recorded before ageing and at certain times after ageing. The property sensor may thereby determine an ageing degree or an ageing status of the fluid.

A property of the fluid may take different values, such as, for example, a property “dynamic viscosity” of the fluid “water” can take values like 1.30 mPa·s or 0.31 mPa·s. Such values are referred to herein as property values. Certain properties may not be related to only numerical property values. A property “curing degree”, for example, may have property values like, for example, “uncured”, “partially cured” or “fully cured”. A property “curing status”, for example, may have property values like, for example, “uncured” or “fully cured”. A fluid according to the present disclosure may be a viscous fluid. Independent of its viscosity, the fluid may be a flowing fluid. The fluid may be a continuously flowing fluid.

“Fluid” or “fluid mixture” are used broadly herein to refer to a composition comprising two or more components. The components may both be liquids, or it may be particulates in a liquid, etc. Generally, a “fluid” or “fluid mixture” refers to a flowable substance. Systems and methods herein may be useful for a range of fluid applications including, but not limited to: paint, resin—for adhesive or other purposes, cure-in-place gaskets, adhesives or other coating materials, dental impression material, void filler, sealant, an engineered fluid, a thermally conductive interface material, a precursor material to any of these, or emulsions or any material that can lose stability over time.

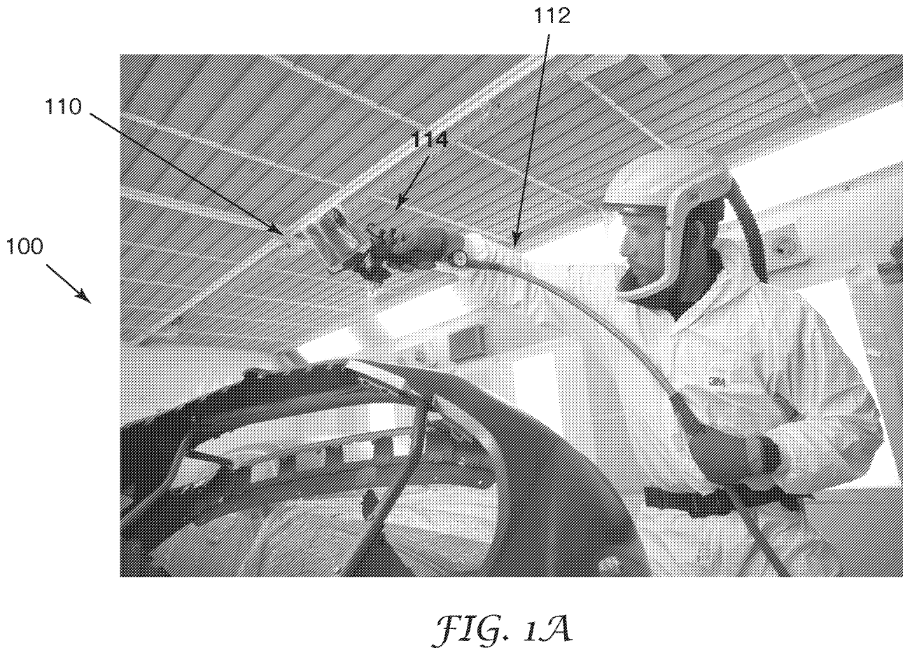

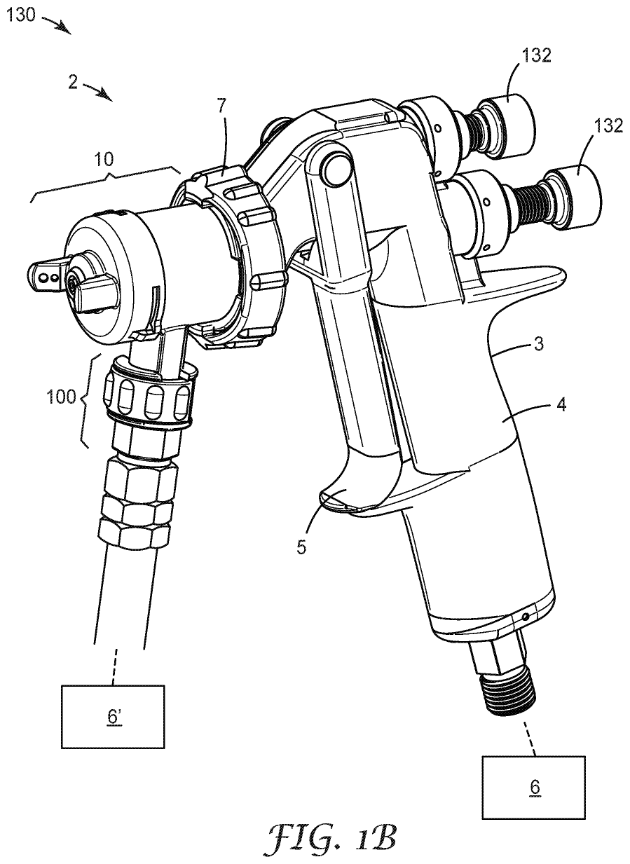

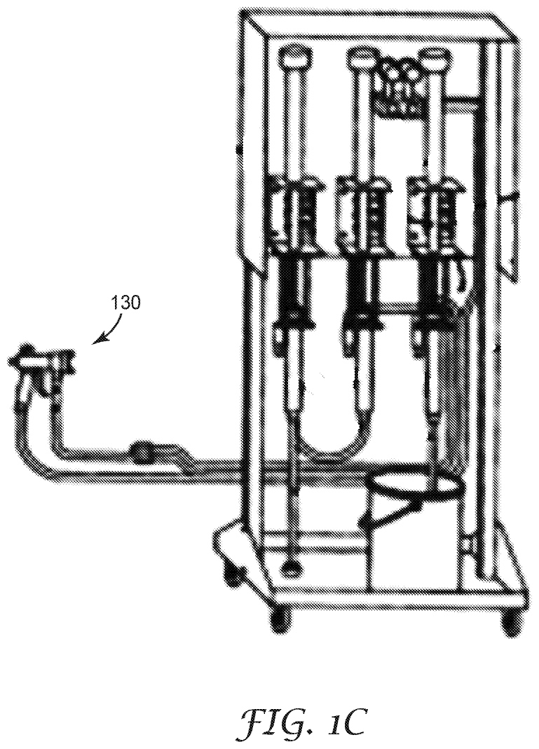

FIGS. 1A-1C illustrate systems for dispensing an atomized fluid that may benefit from systems and methods herein. FIG. 1A illustrates a painting operation 100 where a spray gun 114 atomizes paint from a paint cup 110 using air from air supply 112. However, while a painting operation is illustrated, container 110 could provide other material for dispensing. FIG. 1B illustrates another configuration of a spray gun 130, that receives two materials and provides an atomized mixture. Spray gun 130 may be coupled to a system 150, illustrated in FIG. 1C. System 150 may include pumping systems for one or both components 132, and/or a pressurized air source.

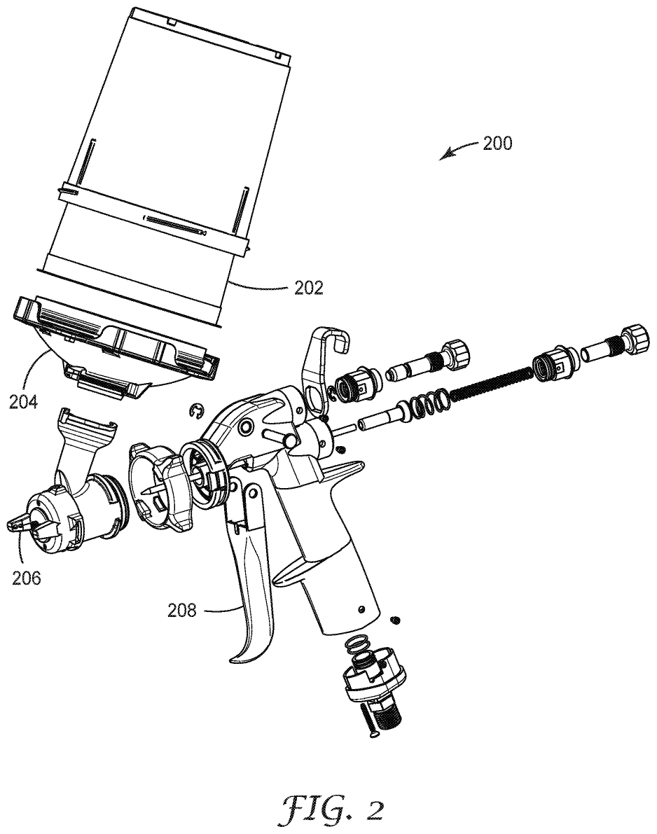

FIG. 2 illustrates an exploded view of a spray gun, in which embodiments described herein may be implemented. Spray gun 200 includes, among other features, includes a container 202 that holds fluid to be dispensed. However, while a container 202 is illustrated that couples to a nozzle using fastener 204, it is expressly contemplated that a larger container, may feed fluid to spray gun 200, for example using a pump. Spray gun may actuate when trigger 208 is pulled, for example.

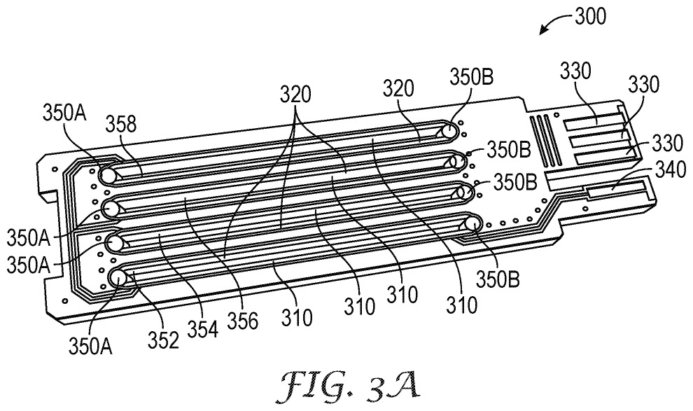

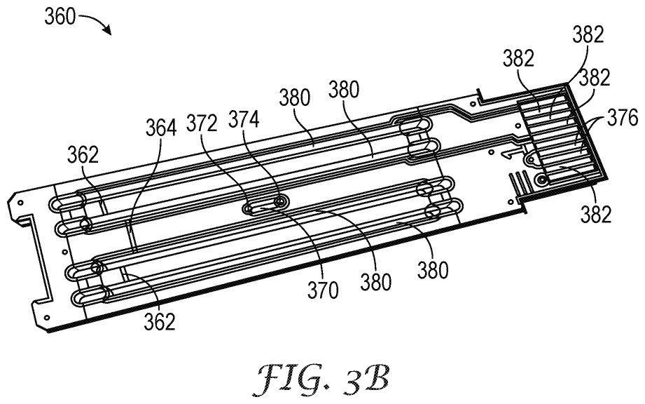

FIGS. 3A-3B illustrate material measurement flow sensors in accordance with embodiments herein. FIG. 3A illustrates a PCB material measurement flow sensor 300. As illustrated in FIG. 3A, a sensing system 300 includes a PCB board 302 with one or more grounds 330 and a TX contact 440. The TX contact provides a transmitting signal to each transmitting electrode 310. Four RX contacts (not shown), located on the reverse side of the PCB, receive the indication of a sensed impedance from each of the electrode pairs. The electrical potential of each receiving electrode 320 is electronically regulated to ground potential separately. The regulator action for each receiving electrode, in some embodiments, is interpreted as an impedance signal for each electrode pair. In the illustrated embodiment, four separate measurements channels can provide information, each through its own TX contact 340 and RX contact (not shown).

In the illustrated embodiment, a sensing system 300 has four electrode pairs, with four transmitting electrodes 310, each paired with one of four receiving electrodes 320. However, it is expressly contemplated that more, or fewer, electrode pairs may be present, depending on available area on a PCB board and sensing needs.

Each of the electrode pairs are decoupled from the adjacent pair such that four separate conductivity measurements are received, one from each electrode pair 310, 320. Sensing system 300 is placed, in some embodiments, perpendicularly to the flow of material, such that a first sensing area 352 receives a first portion of material flow, a second sensing area 354 receives a second portion of material flow, a third sensing area 356 receives a third portion of material flow, and a fourth sensing area 358 receives a fourth portion of material flow. Therefore, system 300 can simultaneously generate four different signals relative to a single material flow, providing a better picture of whether a mixing ratio (or other measured parameter) is consistent across an entire sensing area.

In comparison to previous sensing systems, conductivity measurements required both a positive and a negative pole, which would require two PCBs per electrode pair. In contrast, system 300 allows for four measurements to be taken simultaneously with a single PCB. It also provides a larger surface area for material flow, through a shorter sensor distance.

FIG. 3A illustrates an embodiment where each electrode pair is part of a slot 352, 354, 356, 358. However, it is also contemplated that, instead of being closed on both sides, a sensing area may include a pair of electrodes on a protrusion, or within an aperture, in a “comb”-like structure. However, it may be preferred for both ends to be closed from a structural standpoint, especially with viscous fluids.

As described further herein, the electrodes 310, 320 may be formed by metallization on the interior surface of slides 352, 354, 356, 358, using copper for example. The metallization process may cause electrodes 320 to be connected to electrodes 410. Therefore, a decoupling or disconnecting step is needed. This can be done by breaking the connection, for example by drilling a hole in the positions 350A and 350B as illustrated, by punching out a perforated component, milling, nibbling, etching, laser cutting or another suitable method.

Systems and methods herein may be used for a variety of materials being dispensed. PCB boards often have a maximum operating temperature less than 170 °C, which limits the temperature of materials that can be dispensed through a sensor system 300. Materials may have a range of viscosities, for example up to around 10⁵ Pa s. Higher viscosity might result in a dispensing pressure being insufficient to force the material through slots 352-358 without breaking the sensor. However, higher viscosity materials may be accommodated by increasing the width of slots 352-358. However, sensing system 300 may be less sensitive. Similarly, for materials with particulates, such as suspensions for example, particle sizes have to be smaller than the width of slots 352-358. Additionally, systems herein may be limited to solvents that do not cause corrosion or otherwise damage the PCB 302 or electrodes 310, 320.

FIG. 3B illustrates another embodiment of a sensing system 360, which includes a built-in temperature sensor 370. Temperature sensor 370 sits within a slot with a connection point 372 for a ground signal and a connection point 374 for a temperature signal. Ground signal connection point 372 connects to a ground signal communicator 382. Temperature signal communication point 374 connects to a temperature signal communicator 376. Similar to the embodiment of FIG. 3A, four impedance or conductivity sensor slots 380 are also present, each connected to a ground signal 382. However, it is noted that two different spacings between slots are present in the embodiment of FIG. 3B. A first spacing, 362 is present between a first and second slot 380, and between a third and fourth slot 380, while a second spacing 364 is present between second and third slots 380. Increased spacing 364 may provide improved shielding against interference between electromagnetic fields generated by each electrode pair.

Many mixing processes are at least partially temperature dependent, with material properties like viscosity changing with temperature. Temperature sensors inserted from an external point are often fragile and need to be in the middle of the flow of the material being tested. In the embodiment of FIG. 3B, a temperature sensor is sealed within a housing, which keeps it isolated from the material. The seal layer may be a layer of varnish, for example, which may allow for the thermal contact to be improved relative to other housing materials. As illustrated, the temperature sensor connects via contacts 382 on the edge connector.

FIGS. 3A-3B illustrates an embodiment where slots 352-358, 370 and 380 are ovular in shape, with a generally straight body and rounded ends. However, other configurations are possible. Electrodes 310, 320 may be curved, for example, or otherwise shaped to accommodate an available volume of a dispensing system.

While FIGS. 3A-3C illustrate embodiments where each slot 352-358 comprises a single electrode terminal, it is expressly contemplated that in some embodiments one or more slots 352-358 may house multiple electrode terminals—e.g. with one or more terminals along the length of one or more of slots 352-358. Having a third or fourth terminal may allow for more accurate measurement of an electrical parameter. Instead of a configuration where the circuit configuration includes an ohmmeter that measures a resistance, there is instead an ammeter and a voltmeter. The voltmeter measures a voltage across the circuit, while the ammeter is measuring the current flowing through the circuit. An impedance (e.g. resistance) can be derived from the measured voltage and current. Such a setup may result in more accurate measurements of electrical parameters within a fluid flowing through slots 352-358.

FIGS. 3C-3D illustrate a housing for a sensor in accordance with some embodiments herein. A sensor, such as sensor 300 or 360 may be directly received by a material dispensing system in some embodiments. However, it is also contemplated that, in some embodiments, a housing 390 may receive a sensor 396 directly. Housing 390 includes a receiving slot 392 that receives a sensor, as illustrated in configuration 394.

In some embodiments, housing 390 is built into a dispensing system such that a sensor 396 is received by the dispensing system. In some embodiments, a dispensing system receives housing 390, with sensor 396 already installed therein. Sensor 396 may be sealed into housing 390, in some embodiments, such that a dispensing system receives housing 390.

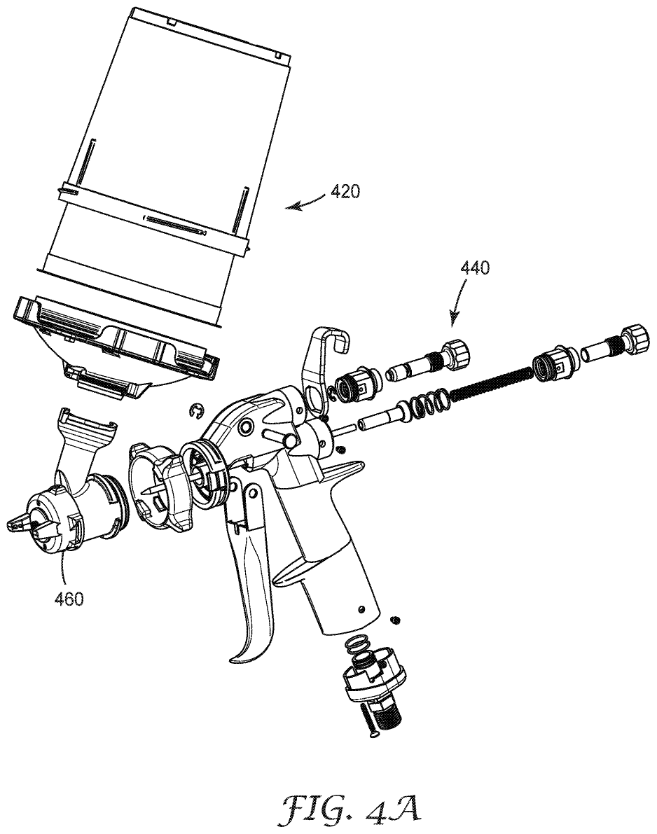

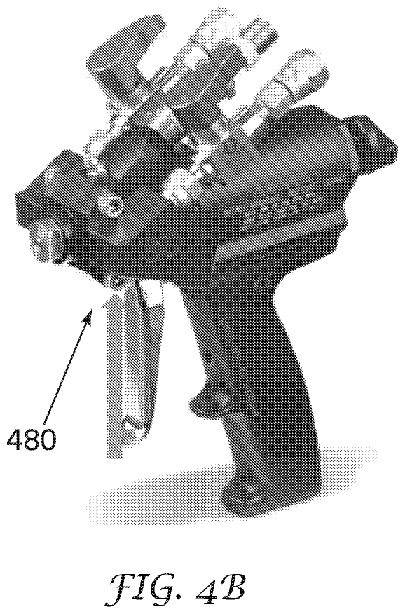

FIGS. 4A-4B illustrate spray guns in accordance with embodiments herein. FIG. 4A illustrates the exploded view of FIG. 2, with potential placement options for a conductivity sensor listed, such as the one described in FIGS. 3A-3D, or any other sensor configurations described herein.

As illustrated in FIG. 4A, one potential placement option is in the container 420, such as in a mixing impeller, in the cup itself, or in the lid.

Additionally, or alternatively, another potential placement option is to place a sensor at a feeding point of the spray gun. In this illustrated embodiment of FIG. 4A, this would provide a quality indication of a mixture as it enters a gun. In a two-part spray gun (as illustrated in FIG. 4B, there could be two sensors at a feed point—one for each incoming material prior to mixing. This may be particularly helpful for troubleshooting if one or both incoming materials are mixtures themselves. Some dispensers described herein benefit from a needle valve 440, which allows for flow regulation.

Additionally, or alternatively, another placement option would be in the atomizing head 460.

FIG. 4B illustrates a 2-part spray gun in accordance with embodiments herein. An additional placement option for a 2-part spray gun would be behind a spray nozzle 480, to ensure that the mixture of the two (or more) incoming components are mixed correctly prior to spraying.

Using systems and methods described herein, it is possible to monitor a number of parameters relevant to the quality of a mixture prior to, or during use of said mixture. Monitoring mixture quality may refer to any of consistency, texture, composition or other relevant quality indication. Sensor systems and methods of use herein may provide indications of mix ratio, curing (e.g. open time, curing speed, temperature changes) and may provide in-situ process indications such as aging, air bubble detection or concentration, lot-to-lot variation, raw material quality, and phase separation. Using sensor systems herein, it is possible to automatically detect a quality concern (mix ratio imbalance, phase separation, etc.) and provide indications for correcting the quality concern, so that a correction can occur in-situ.

Early detection of quality concerns can help reduce correction time and will, therefore, reduce operation time, corrective supply cost, and corrective operation time and cost. Sensors described herein can be implemented in many parts of a dispensing operation-at intake, during or after mixing, within a dispenser, within a container, etc.

Sensors described herein are communicable with a computerized control system which may provide an alternating current (AC) voltage to generate a required electric field needed for measuring conductivity, impedance or dielectric constant using a suitable sensing system, such as that described herein. The control system may also, in some embodiments, provide a current. While various examples of this disclosure are described with respect to the use of AC, it will be appreciated that techniques of this disclosure may be performed using direct current (DC) in other examples.

When running an actual measurement of quality control parameters of a mixture, the measured impedance responses (MIR), each measured at certain measurement sensing frequencies (MSF), can be recorded in the control system.

In order to derive a value for the mixing ratio, for example, from the measured impedance responses at the measurement sensing frequencies, software running on the control system identifies, within the set of calibration impedance response triples, those triples having the closest calibration response impedances, closest to the measured impedance responses, and the closest calibration sensing frequencies, closest to the measurement sensing frequencies. This identification and a potential interpolation can be performed easily by using the parametrized multi-dimensional polynomials modelling the plurality of data sets, i.e., the plurality of triples of (CMR, CSF, CIR). From those calibration data, the software derives a value for the (so far unknown) mixing ratio in the actual measurement.

The same sensing frequencies used for calibration will often be used also for the measurement. There may, however, occur a mixing ratio in the measurement for which no calibration impedance response had been determined in calibration. So there may be not an exact match in both sensing frequency and response impedance between a triple in the calibration data set. In such a case, an interpolation between two suitably chosen calibration triples, containing two calibration impedance responses close to the measured response impedance, yields an interpolated calibration mixing ratio which can then be considered the mixing ratio in the measurement. The interpolation is performed by software on the control system 220, using the parametrized multi-dimensional polynomials.

The result of the interpolation and derivation is a value of the mixing ratio of components A and B as the mixture passes through the PCB sensor during the measurement.

In the present embodiment, the calibration impedance responses were measured in their dependence on two parameters, namely on the sensing frequency and on the mixing ratio. In other embodiments, dependence of impedance responses on further parameters may be taken into account, such as, for example, dependence on the temperature of the adhesive in the sensing zone. A data set of the calibration impedance responses would then be a quadruple of values, such as (CMR, CSF, CIR, Temperature), and the pre-stored set of calibration impedance responses would be a set of quadruples forming a four-dimensional data field, which is specific for the mixture. Taking further parameters into account could make a data set be a quintuple of values, or high-order tuples of values, so that the data sets of calibration impedance responses is a multi-dimensional data field of more dimensions and can be represented by different parametrized multi-dimensional polynomials.

A control system may record the values for mixing ratio, with a time stamp, for quality assurance. The mixing ratio derived during the actual measurement can be checked continuously against a desired mixing ratio. If its deviation from the desired mixing ratio is larger than acceptable, the control system may change the flow rate of either components suitably to adjust the measured mixing ratio towards the desired mixing ratio.

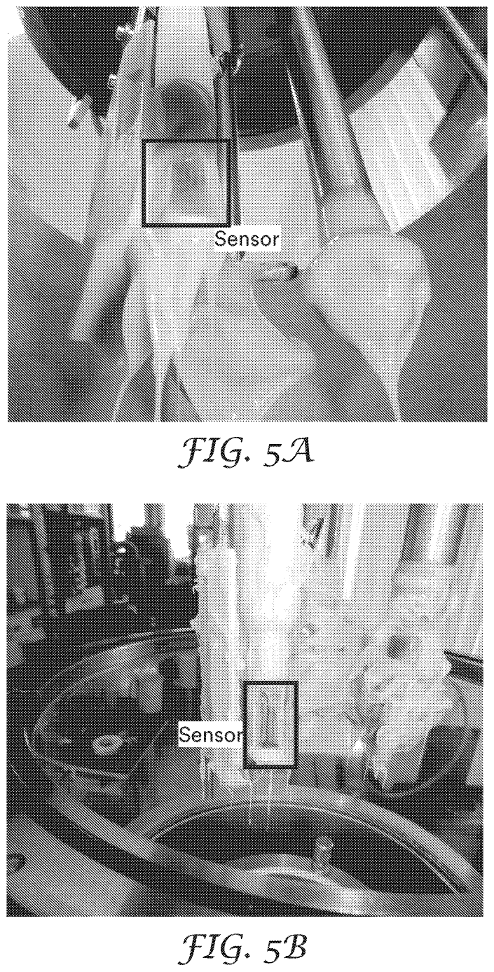

A method of forming sensor systems like those illustrated herein may be similar to that described in PCT/US2022/052343, for example FIG. 5 and the associated description, which is incorporated herein by reference.

FIGS. 5A-5B illustrate material measurement flow sensors as used in accordance with embodiments herein. As illustrated in both images, sensors according to embodiments herein can be placed in direct contact with a material or fluid, providing a conductivity measurement based on that direct contact. This provides a more accurate measure of mixing ratio than other methods that do not allow for direct contact between a sensor and a material. However, as illustrated in FIGS. 5A and 5B, a sensor is coated with material after use. In scenarios where the material of interest is corrosive, highly viscous or is curable, it is beneficial to be able to discard the sensor after use.

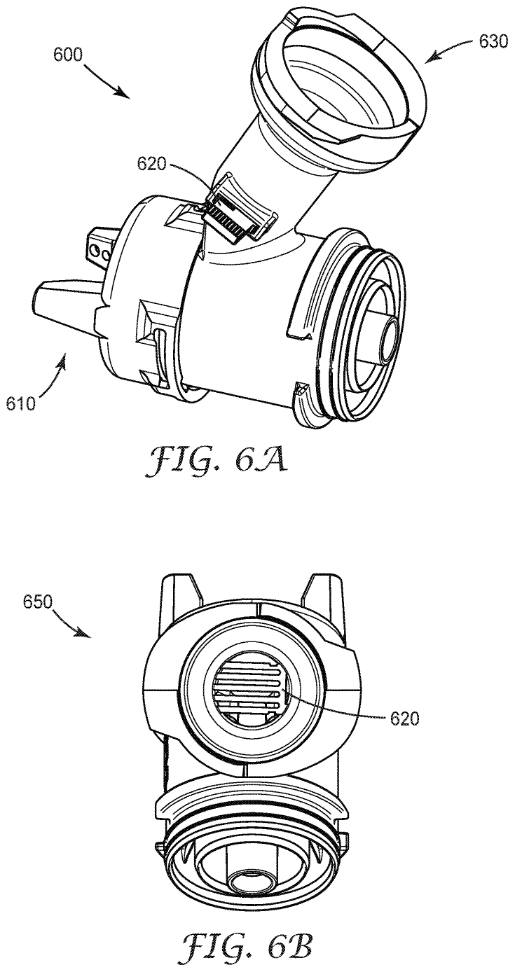

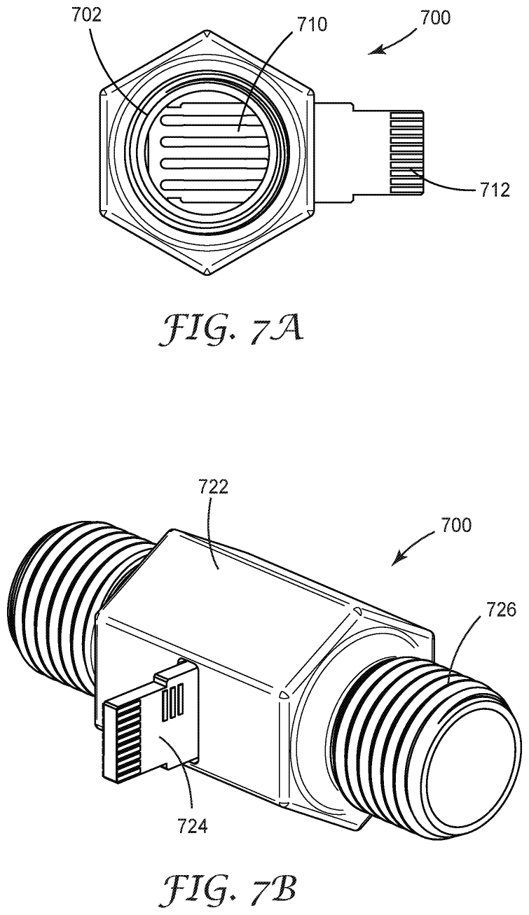

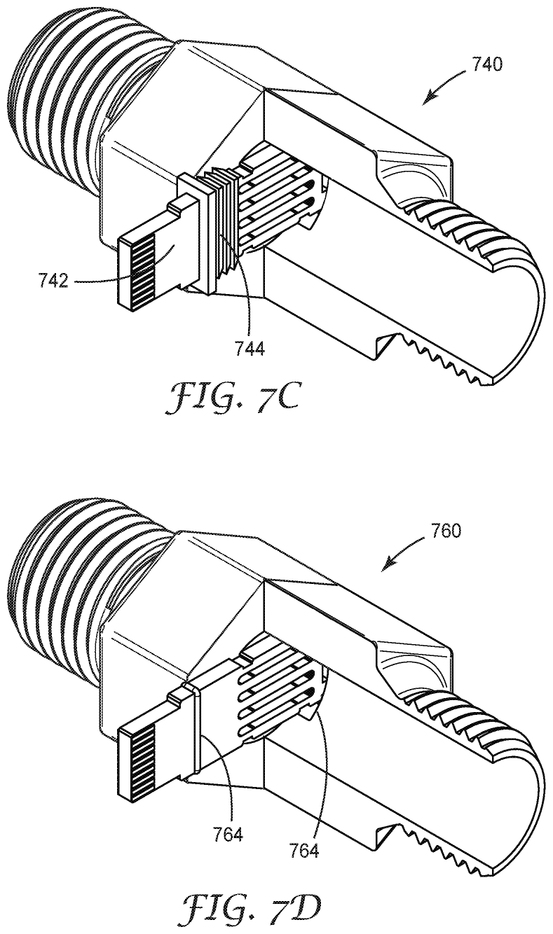

FIGS. 6-7 illustrate example implementations of dispensing systems with sensor systems installed in accordance with embodiments herein. FIGS. 6A-6B illustrate views 600, 650 of a PCB sensor 620 incorporated into a nozzle such that fluid flow from a container (not shown) passes from adapter 630, through sensor 620, and then is dispensed by atomizing nozzle 610.

FIGS. 7A-7D illustrate placement of a sensor within a conduit. FIG. 7A illustrates a sensor 710 within conduit 700. Sensor 710 has four electrode pairs, each placed within slots such that, as a mixture passes through conduit (into the field), it is forced through the slots of sensor 710, contacts each electrode pair, and sensed conductivity measurements are passed to a control system, by edge connector 712, for example. Variation in the conductivity measurements between one electrode slot and another electrode slot indicates may indicate a variation in quality consistency of the mixture.

FIG. 7B illustrates a perspective view 700 of a conduit 722. Conduit 722 may couple to another part of a dispensing system or a fluid transport system. Conduit 722 may couple to another part of a fluid flow system using threading 726, or another suitable fastening system.

FIGS. 7C and 7D illustrate cutaway views of a conduit. In FIG. 7C, an over-molded plastic 744, 740, 760 is used as a seal to hold a PCB sensor in face. Such a seal may have an end stop to confirm the sensor is in place. However, other seal options, and position confirmation options (e.g. a snap or clip) may also be possible. The illustrated seal may include barbs to maintain a connection.

In FIG. 7D, a different seal configuration is illustrated—an O-ring can be used. Corresponding recesses that can receive an O-ring 764 may be machined into the conduit to stabilize the sensor against the pressure of fluid flow.

The illustrated conduit may be replaceable, such that a sensing assembly is a single-use assembly, in some embodiments. In other embodiments, the sensor is removeable such that the PCB sensor is a single-use sensor.

The exemplary embodiments illustrated in FIGS. 7A-7D concern a PCB-based impedance sensor that can be attached to a static mixer, using an adapter or other connection mechanism, providing mix ratio information in real-time. The use of an adapter that can receive a PCB unit allows for compatibility of the PCB sensor with a number of dispensing systems.

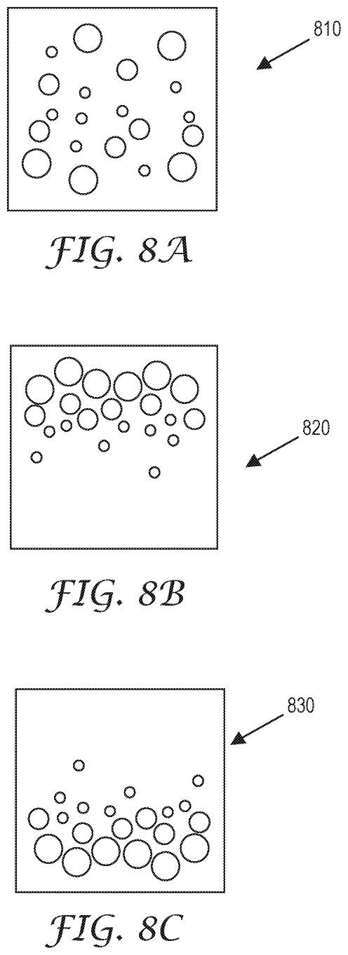

FIGS. 8A-8C illustrate three dispersion states that may be detected using sensor systems and methods described herein. FIG. 8A illustrates a stable dispersion 810 where particles (or another fluid) are dispersed evenly throughout the mixture. From a colloidal point of view, a dispersion is stable when flocculation of separated particles is prevented because the particles repel each other. FIG. 8B illustrates an example of a dispersion 820 experiencing creaming, where separation is occurring such that one material is separating to the top of the mixture. A dispersion 830 is illustrated in FIG. 8C, which is experimenting sedimentation, where particulates are settling at the bottom of a container.

Described thus far is a sensor system that can be used for evaluating the quality of a mixture during a dispensing operation. However, it is expressly contemplated that the same, or similar sensors, may be used to evaluate a mixture in a container. For example, a painting operation often involves mixing different fluids, or mixtures, into a container prior to coupling that container to a dispenser.

Additionally, many materials may be stored in large containers prior to use, and those containers may not be clear or otherwise allow for easy visual inspection. For example, many materials are stored in 55-gallon drums prior to use, which are not transparent. It is difficult to visually confirm settling, or whether a mixture is close to a phase separation.

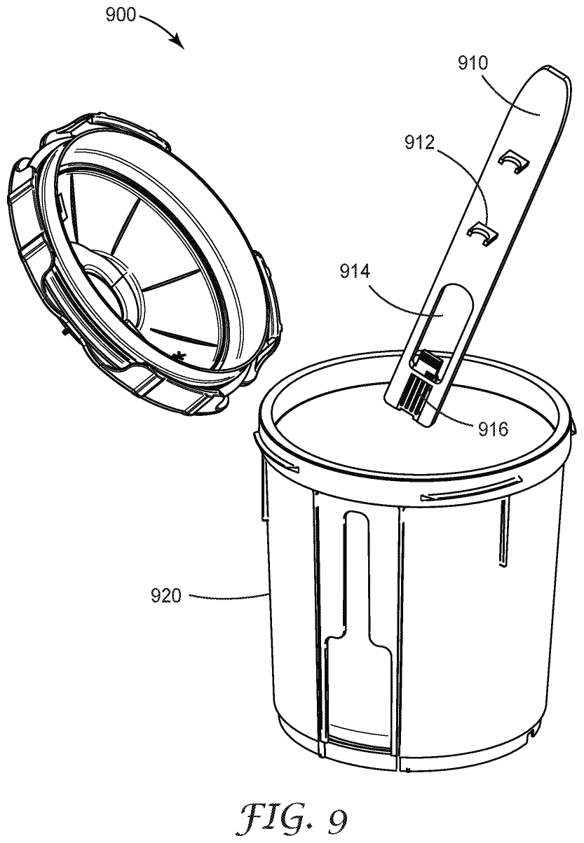

FIG. 9 illustrates an embodiment of a stir stick configured to provide in-situ conductivity measurements for a mixture. Schematic 900 illustrates a stir stick 910 that may be used with a container, such as paint mixing cup 920. However, stir stick 910 may be suitable for other containers, and other mixtures as well.

Stir stick 910 provides a sensor 916 that can be moved through a mixture (or placed in a flowing mixture). A window 914 is included in stir stick 910 to allow for connection of an edge connector to wire leads. However, in some embodiments a wire lead may connect to edge connector in another suitable manner. In some embodiments, stir stick 910 includes one or more retention clips 912, or other suitable wire retention features that assist in coupling an edge connector of sensor 916 to wire leads.

Sensor 916 is illustrated as coplanar to stir stick 910. This may allow for sensor 916 to be more easily cleaned after a stirring operation (e.g. by wiping down stir stick 910). However, it is expressly contemplated that sensor 916 and/or stir stick 910 may be single-use products such that they are discarded in between uses.

Sensor 916, in other embodiments is offset from stir stick 910 (e.g. mounted to a first side or the other side) such that wire leads may be connected without a window 914.

Stir stick 910 is configured such that, as it is moved relative to a mixture, the mixture is forced to flow through slots in sensor 916.

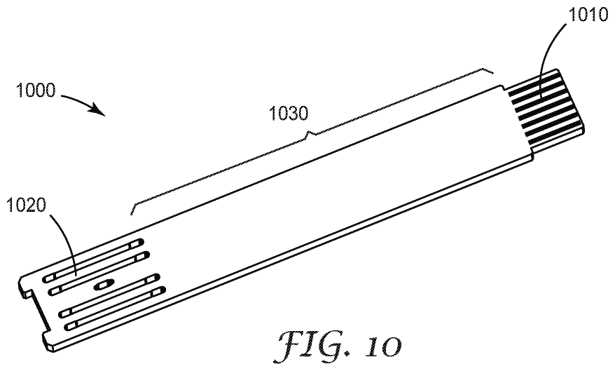

FIG. 10 illustrates an extended sensor in accordance with embodiments herein. Sensor 1000 is illustrated in FIG. 10 as having a length 1030 of separation between an electrode portion 1020 and edge connector 1010. Edge connector 1010 should not come into contact with a mixture. Therefore, having a separation 1030 in between edge connector 1010 and electrode portion 1020 increase the flexibility of use for a conductivity sensor—for example allowing sensor 1000 to be used in a deeper container to ensure consistency throughout the depth of the container. Sensor 1000 can be dipped and used to stir the sensor inside a mixture without the edge connector to touch fluid (and short circuit), allowing for material characterization data (conductivity, temperature and dielectric constant) to be monitored and visualized in real-time.

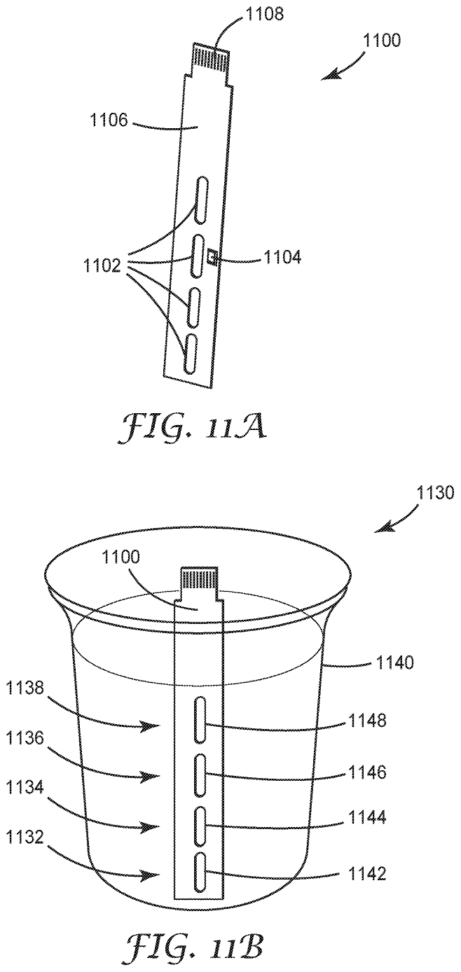

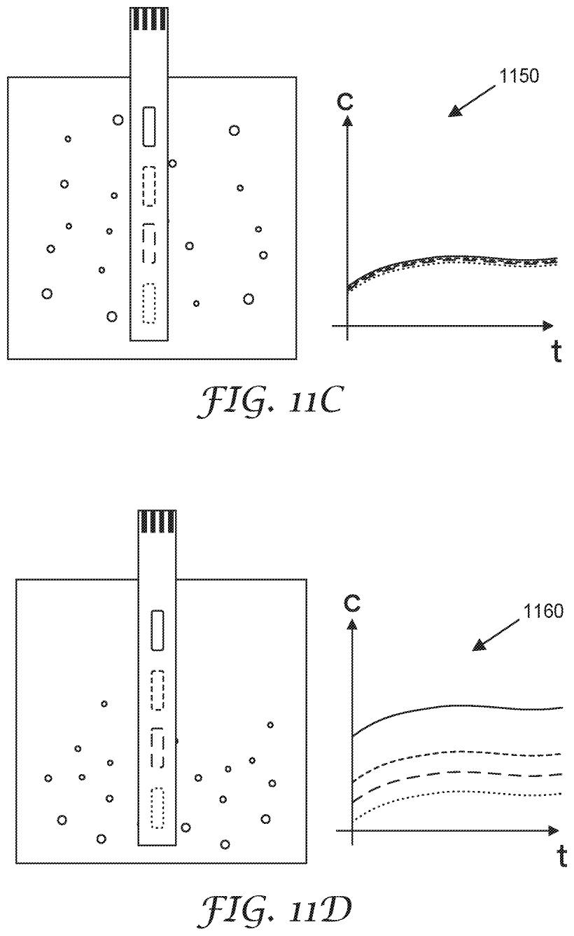

FIGS. 11A-11D illustrates a sensor with electrode in a series configuration in accordance with embodiments herein. Discussed thus far have been sensor configurations where electrode slots 1102 are coplanar along an edge of the sensor opposite edge connector 1108. A temperature sensor 1104 is included on the PCB board as well. A length 1106 between edge connector 1108 and the electrode slot 1102 closest to edge connector 1108 is also included to reduce the chance of edge connector 1108 contacting fluid in a container.

FIG. 11B illustrates a scenario 1130 showing the use of sensor 1100 in an incomplete mixture in a container 1140. The arrangement of sensors in a vertical stack along a PCB allows for each electrode slot to be positioned at a different depth within container 1140. Electrode pair 1142, at a lowest depth measures a first conductivity at depth 1132. Electrode pair 1144, at a second-to-lowest depth measures a second conductivity at depth 1134. The conductivity at depth 1132 will differ from the conductivity at depth 1134 because the composition is different. Similarly, a conductivity measured by electrode pair 1146, at depth 1136, will be different than a conductivity measured by electrode pair 1148, at depth 1138, because the concentrations differ.

While FIGS. 11A and 11B illustrate a sensor 1100 with four electric pairs arranged in a vertical stack on a PCB, it is expressly contemplated that a different number of electrode pairs may be present, for example only 2 electrode pairs, only 3 electrode pairs, or more than 4 electrode pairs, such as five electrode pairs, six electrode pairs, or more than six electrode pairs. Additionally, a spacing is present between electrodes, which may be longer or shorter than that illustrated.

In some embodiments, sensor 1100 includes only one electrode pair. One electrode pair may, for example, be useful for measuring an ongoing mixing process.

A sensor such as sensor 1100 may be particularly useful for containers housing dispersions or emulsions that, currently, need to be continuously rotated, or constantly in motion, to prevent sedimentation or creaming. However, the resulting mixing quality is unproven. Sensor 1100 may be used to measure current dispersion/emulsion consistency or built into a stir stick or other stirring implement such that in-situ mixing indicia can be provided to ensure that a mixture is sufficiently mixed, but that time is not wasted over-mixing.

FIGS. 11C-11D illustrate example mixtures and resulting conductivity measurements using a sensor 1100. FIG. 11C illustrates a stable dispersion 1150, which results in conductivity measurements from each electrode pair that are very similar. FIG. 11C illustrates a dispersion 1160 experiencing sedimentation, which causes conductivity measures to differ between the different electrode pairs as the concentration varies with the depth of electrode pairs in a mixture.

Sensors like those illustrated in FIGS. 11A-11D may be particularly useful for measuring flocculation or aggregation in-situ, potentially before significant sedimentation or phase separation has occurred. This may help ensure that corrective action is taken sooner.

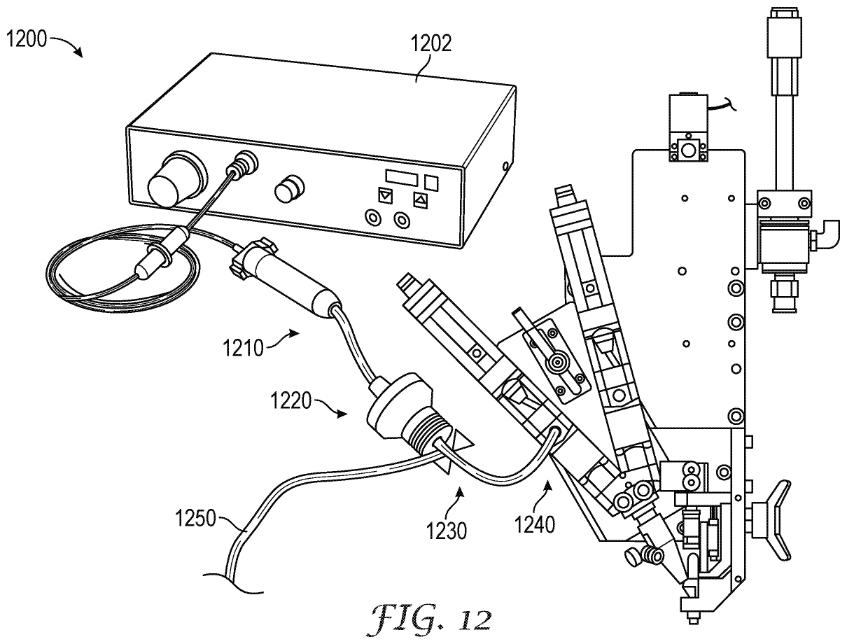

FIG. 12 illustrates another embodiment of a system in which embodiments herein may be useful. System 1200 illustrates an exemplary dispensing system 1200 with a controller 1202, which may include a motor that provides pressure for dispensing a mixture through dispenser 1210. The mixture being dispensed may have a quality control issue not readily apparent to an operator. Systems and methods herein may be useful for detecting and correcting a quality control issue. As described above FIGS. 7A-7D, sensors described herein may be placed within a conduit, e.g. sensing area 1210 where a material is first provided. As a material flows through, or exits, sensing area 1210, it may have entrained air bubbles or droplets formed of only one component (or a subset of components) of the mixture. If the material coming from sensing area 1210 is provided for combination into a mixture downstream, air bubbles also will affect the resulting mix ratio. For a dispensed mixture, this can result in an incorrect mixing ratio being dispensed. For viscous fluids, this may result in an area of a worksurface not receiving dispensed fluid.

Similarly, an improperly mixed mixture also present quality control concerns. Droplets may indicate that a phase separation is imminent in a mixture.

It is important to detect, and potentially remove, air and/or droplets from a dispensing system. Therefore, in some embodiments, before reaching a dispenser 1240, material passes through sensing area 1210, which includes any of the PCB-based sensors described herein. The sensor detects an inconsistent mixture (e.g. air bubbles or droplets) such that a corrective action mechanism 1220 may be activated before the material reaches dispenser 1240.

For entrained air bubbles, a valve 1230 can be opened, allowing for the air bubble containing portion to leave through stream 1250. When the air bubble has passed, valve 1230 closes, and the material continues on to dispensing system 1240.

Similarly, if phase separation, or other quality issue is detected in a mixture passing through sensing area 1210, valve 1230 may divert the mixture in flow 1250 for corrective action—e.g. re-mixing, degassing, excess purge, etc. Phase separation may be detectable as droplets of a first material begin to form from aggregation.

Controller 1202 may also cause dispenser 1240 to stop dispensing until the mixture is deemed sufficiently mixed. In the illustrated embodiment, the material from flow 1250 may be returned through controller 1202 to sensing area 1210 to confirm quality is sufficient to resume dispensing. Controller 1202 may provide a notification to other systems that are connected to dispensing system 1240 (e.g. a motion controller that moves 1240 relative to a surface receiving dispensed material) and/or to an operator through a user interface on a display, an audible indicia, etc.

Valve 1230 automatically opens and closes, in some embodiments, based on an indication from either sensing system 1220 directly, controller 1202, or another controlling system that, based on a conductivity measurement received from system 1220, sends a command to implement a corrective action.

FIG. 12 is a schematic of a system 1200 with components for one material line clearly illustrated. However, it is expressly contemplated that, as illustrated, a dispensed mixture may be formed from two components, and a similar system may be provided for the second component.

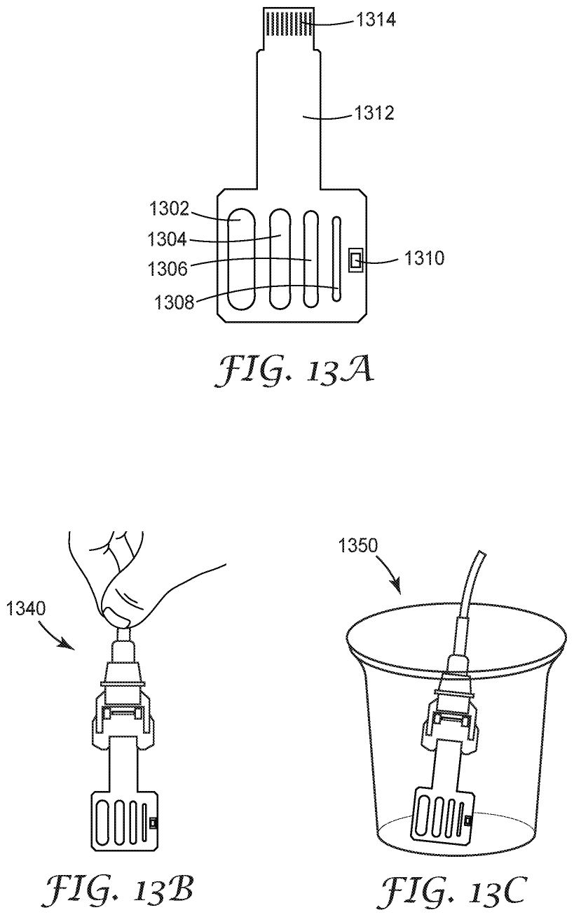

FIGS. 13A-13C illustrate a sensor configuration that may be particularly useful to detecting bubbles or aggregation of droplets of a second phase forming prior to a phase separation.

FIG. 13A illustrates a dip sensor that may be particularly useful for detecting air bubbles or droplets in a mixture. Sensor includes four electrode pairs in four slots, 1302, 1304, 1306 and 1308, that are different sizes. Slot 1302 is wider than slot 1304, which is wider than slot 1306, which is wider than 1308. Slots 1302-1308 are illustrated in an arrangement from thickest to thinnest, however it is expressly contemplated that other arrangements are possible. Similarly, while four coplanar electrode pairs are illustrated, all substantially the same distance from a connection end of a sensor—e.g. the portion that connects to a signal reader. An edge connector 1314 is shown as one example, however other suitable connections may be possible.

Slots 1302-1308 are designed to both detect bubbles or droplets and provide an indication of size. Generally, a consistent mixture with no bubbles or droplets provides an insulation effect, and maintain a consistent conductivity across all electrode pairs. When a droplet reaches a width of one of the slots, the droplet will connect both sides of the electrodes, resulting in a detectable change in conductivity.

The design illustrated in FIG. 13A is illustrated as having slot widths that linearly increase—e.g. 1 mm, 2 mm, 3 mm and 4 mm. A linear increase in diameter corresponds to a cubic increase in volumetric flow through the apertures. Such a configuration provides a good understanding of how quickly phase separation will occur and/or how stable a mixture is. For example, if phase separation is more than a hour away, it may still be possible to dispense a mixture without corrective action.

However, it is explicitly contemplated that some embodiments may require smaller or larger slot sizes. For example, a thinnest slot may be as thin as 100 μm, or thinner than 150 μm, or thinner than 200 μm, or thinner than 300 μm, or thinner than 400 μm. One or more slots may be thinner than 500 μm. One or more slots may be thinner than 1 mm. For other applications, for example a stir stick being used in a larger measurement operation, such as checking a mixture quality of a 50 gallon drum. For example, larger slot sizes may be useful, such as slots that are at least 5 mm, at least 10 mm, at least 15 mm or even at least 22 mm in size.

In addition to the change in width, the slots may also change in length to suite a particular application. For example, in checking the shelf-life of a larger container, the overall sensor may need to be much longer for example up to, or over, 1 meter in length. In such instances, apertures must be larger-both to increase signal strength and to allow for significant flowthrough. A length may be increased to increase signal strength, balanced with a width selected to allow flowthrough without sacrificing signal strength. For example, for a meter-long sensor, the dimensions may be 10 centimeters long and 1 cm wide.

E.g. when a droplet (or bubble) reaches a diameter as wide or wider than slot 1308 (the narrowest slot), it connects the two electrodes within slot 1308, resulting in a conductivity spike only for electrode pair 1308, as, until the droplet (or bubble) diameter grows to a width as wide or wider than slot 1306, it will not connect the two sides of slot 1306. The conductivity will return to a baseline for the mixture once the bubble (or droplet) passes through slot 1308. Depending on the number of droplets (bubbles) in the mixture, the conductivity spike frequency changes.

Sensor 1300 also includes a temperature sensor 1310, illustrated as in-line with electrode pairs 1302-1308, it is expressly contemplated that temperature sensor 1310 may be positioned in another suitable position. Additionally, it is contemplated that, for some embodiments, a temperature sensor 1310 is not needed, e.g. for a mixture that does not change viscosity significantly over a temperature range of use.

Sensor 1300 is also illustrated as having a length 1312 that separates the edge connector 1314 from electrode pairs 1302-1308. However, length 1312 may not be necessary if sensor 1300 is used as an in-line flow sensor, for example mounted within a conduit as illustrated in FIGS. 7A-6D.

FIGS. 13B and 13C illustrates a sensor connected to lead wires 1340, illustrating how a length 1312 provides additional separation from electrode pairs 1302-1308 when in use in a container 1350.

Described herein thus far have been a number of sensor configurations where a single line of parallel electrodes is illustrated (e.g. in the horizontal configuration of FIGS. 3-7 and 13, or a vertical configuration such as FIG. 11). However, it is expressly contemplated that an arrangement that combines the features of both configurations is possible. For example, a grid of electrode pairs may be useful to detect consistency at multiple depths as well as mixing quality (or the presence of droplets/bubbles) simultaneously. Additionally, while embodiments herein illustrate sets of four electrode pairs in different configurations, it is expressly contemplated that more, or fewer, electrode pairs may be present in any vertical or horizontal arrangement. Described thus far herein are embodiments of sensors formed from a PCB, designed to receive a fluid flow through apertures therein. However, it is expressly contemplated that sensors herein may take other shapes and configurations.

Flexible sensor configurations may be used in embodiments herein.

An embodiment wherein a sensor can be adhered to a surface. An adhesive backing is adhered to a flexible substrate, opposite an electrode. In some embodiments, electrode includes a gold conductive pattern. However, other materials may be suitable for some applications. A reader connection is present on one end of sensor. Connection may be an industrial edge connector, or a data transmitter such as an NFC or RFID tag. For some applications, a low power wireless solution is preferred.

Two sensors may be placed opposite each other. A transmitting electrode placed opposite a receiving electrode allows for electrical parameter signals for a material flowing in direction when a voltage or current is transmitted by electrode, creating electric field. Four separate sensing areas may be on a receiver, such that four sensor signals can be transmitted, providing a better understanding of material flowing between electrodes.

Another flexible sensor configuration may include two electrodes that can be positioned in a flat configuration, or a parallel configuration, to enable bulk sensing measurements as fluid passes between electrodes. However, as electrodes are printed on a flexible backing, it is expressly contemplated that other configurations may be possible. For example, flat configuration may be useful for obtaining a surface-sensing measurement. Additionally, electrodes may be rolled into a channel sensor having an circular, ovular or polygonal shape, as illustrated in FIGS. 7-9 and the associated description of PCT/IB2023/062401, incorporated herein by reference. Such a configuration may be suitable for use in a static mixer.

Another flexible sensor configuration includes sensing surface of a sensor may utilize surface-sensing techniques to provide a sensed electrical property signal to a reader using an edge connector. Other data transfer mechanisms are envisioned such as an NFC or RFID tag.

The flexible sensor configuration may include a simplified electrode configuration for surface sensing. An interdigitated comb structure, with transmitting electrode portions interleaved with receiving electrode portions. Transmitting electrode portions generate an electric field on a surface of sensor, and receiving portions sense a signal, which is reported by edge connector to a signal reader. However, while an edge connector is illustrated, it is expressly contemplated that other suitable data transfer options can be used.

Flexible sensors can be formed using a number of suitable technologies. Printing techniques can be used to print patterns on a number of material substrates, flexible or rigid, such as thin film transistors, capacitators, coils, resistors, etc. Printed electronics offer significant opportunities for low-cost electronics with simpler fabrication. However, printed electronics may only be suitable for applications where low-performance is acceptable.

Another technique that may be suitable is in-mold electronics. Electronics may be printed using functional inks on a moldable substrate, such as PET or another suitable substrate. The substrate is then thermoformed into shape. Once the electronics are formed (e.g. a single surface sensing device or two bulk sensing devices), a sensor is assembled such that voltage or current can be applied to a transmitting electrode and a signal received from a receiving electrode.

Laminated structures consist of at least one non-conductive, or insulating, layer. In embodiments where the electronics are integrated onto a surface of a standard component—e.g. a molded material, a composite, etc. In such embodiments, the non-conductive layer may be formed of durolastic materials. One suitable durolastic material may include an FR-4 epoxy. However, other suitable materials may be used. In some embodiments, the non-conductive layer may be formed from a polyamide, a polycarbonate, a polypropylene, a phenolic material, ABS or another suitable material. In accordance with embodiments herein, the non-conductive layer may be modifiable to receive solder. In accordance with embodiments herein, the non-conductive layer must be modifiable to receive a conductive material—e.g. through metallization or another suitable process.

As described herein, laminated structures can be formed of a number of suitable materials. In some embodiments, laminated structures are formed using additive or subtractive manufacturing techniques. Such “printed” materials may allow for embodiments herein to be implemented in a number of additional configurations.

In some embodiments, laminate structures can be formed using classical additive manufacturing techniques—e.g. Fused filament fabrication, SLA, IJ. Non-conductive materials for such embodiments may include SLA/SLS materials, which may be UV-curable, for example. Sintermaterials, such as PA or ceramics may also be used. Fused filament fabrication materials, such as ABS or another suitable material, may also be used.

Conductive materials for laminated structures may include a base material with a surface finishing, in accordance with embodiments herein. The base material may be copper, for example. Surface finishing materials may include nickel or gold. Liquid inks may be used, and may contain silver or graphite materials. In some embodiments, nanomaterials such as graphene or carbon nanotube-based conductive inks or sprays may be used. Silver chloride may be used, for example. Carbon inks may be used, in some embodiments, either alone or as a complement to a conductive silver inks. Carbon inks may provide lubricity, protection of the silver surface and prevention of silver migration. Some conductive inks may include, for example: AgNW, AgNP, AuNP, CuNW, CuNP, PdNP, or a mixture thereof.

Dielectric inks may be used in some embodiments herein to print dielectric layers, conformal coatings and/or encapsulations. Non-conductive, dielectric inks may insulate multilayer circuitry to allow for circuitry crossover and multilayer applications. Dielectric inks offer flexibility, humidity resistance and improved strength.

Resistive inks may be used in accordance with some embodiments herein. Resistive inks may be based on blends of silver, carbon and non-conductive pigments to adjust resistance levels for printed resistors, potentiometers and heating elements.

In some embodiments herein, 3D electronic printing techniques are used, such as piezo/valve jet, aerosol based jetting, multinozzle ink jetting, 3D dispensing, printing/laser ablation, pneumatic spraying, and/or US spraying.

Using 3D printing techniques, it is possible to form sensors described in embodiments herein directly onto a surface that contacts a fluid—e.g. into a conduit, a dispenser, a mixing unit, a container, etc. A greater range of functional elements, such as flexibility, is possible. Electrically functional inks are deposited on the substrate, which results in active or passive devices.

For example, a conduit could be formed with a conductive pattern that allows for measurement of an electrical parameter. The conduct may include multiple sensing areas along its length to track electrical parameters as a fluid passes each sensing area.

The printed electronics may be printed directly into a housing, a conduit, a container, a 2K cartridge, a static mixer, etc. For example, a housing may be formed from two components, one having the transmitting electrode, the other having the receiving electrode. One component, or another component may include the edge connector or another suitable data transmitter.

Sensors and sensing systems herein may be useful for a number of quality control applications—for flowing material or static material. Devices have points of failure—as components wear and tear due to use, the risk of device failure increases. When failure occurs, maintenance is needed.

When failure is not detected, reactive maintenance is needed to repair or replace a failed component. Preventative maintenance can be taken when signs of failure are detected before failure occurs. Predictive maintenance can be taken before damage occurs.

One example where sensors herein may be useful is the manufacture and maintenance of electronic-vehicle batteries. Batteries and their housings include many conductive materials sealant, filler, material separating battery cells, etc. In one embodiment, the housing may include thermoformed sensors like those described herein may, when a housing is sealed, form an electronic circuit that can be used to detect conductivity of any material that contacts the housing. Alternatively, a sensor with a flexible backing may be placed within the housing where fluid will contact it. If the sensor signals are not as expected, an error in manufacturing may be corrected before the battery is placed in a vehicle. Additionally, a sensor placed inside an installed battery may report signals during use, and could be used as a way to determine if a recall is needed or whether maintenance is required. The sensor may have a data transmitting device that operates wirelessly, and associates the sensed signals with a vehicle ID. Health monitoring may also be useful for applications outside electronic vehicles, such as in aerospace manufacturing, etc.

Described herein thus far are sensor systems that are based on a single PCB board. Such systems are relatively inexpensive and, therefore, cost effective to use and replace. However, one disadvantage of designs described thus far is the large stray field compared to the main field present between each electrode pairs. The stray field effect is caused by the short distance between material flow input and output, e.g. the thickness of the PCB. One way to reduce the stray field effect is to solder multiple PCBs, each with electrode-containing apertures, into a PCB stack.

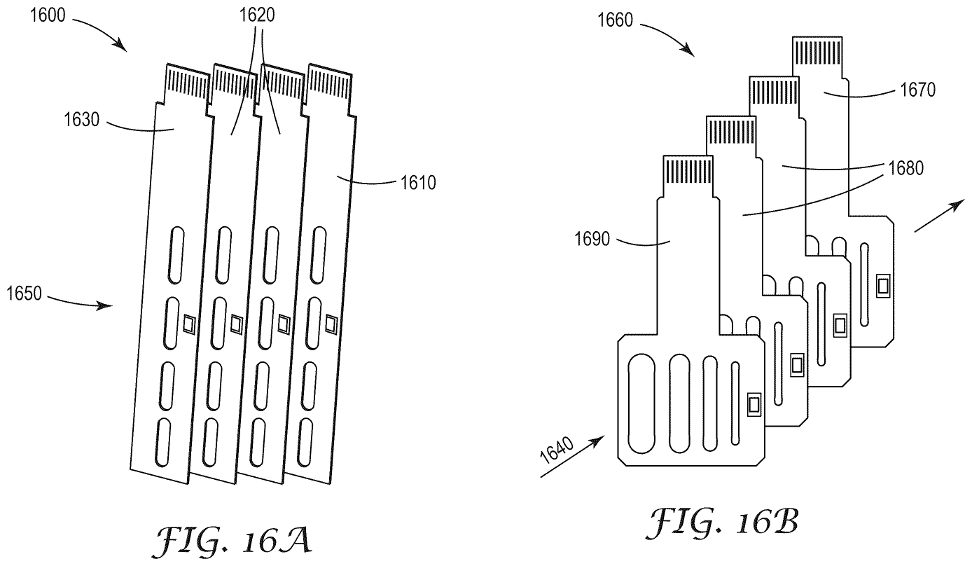

FIG. 16A-16B illustrate a stacked PCB sensor in accordance with embodiments herein. FIG. 16A illustrates a stacked PCB sensor of PCB boards similar to FIGS. 11A-11B, while FIG. 16B illustrates a stacked PCB sensor of PCB boards similar to FIGS. 13A-13C. As illustrated, in one embodiment, sensor stack 1600 may include four PCB sensors, with one 4-layer PCB 1610, two stacking PCBs 1620, which are provided to get the required sensitivity by increasing the electrode surface area, and a top PCB 1630. In some embodiments, flow through sensor stack 1600 is indicated by arrow 1650.

Similarly, sensor stack 1660 illustrates a stack of four PCB sensors, with one 4-layer PCB 1670, two stacking PCBs 1680, and a top PCB 1690. Flow through sensor stack 1660 is indicated by arrow 1640.

While the embodiment of FIGS. 16A-16B both illustrate a four-layer sensor stack, it is expressly contemplated that fewer, or more PCB sensors, may be coupled together. For example, as few as two PCBs or as many as five, six, seven, eight, nine, ten or more PCBs.

Each stacked sensor 1600, 1660 provides the benefits of a single PCB sensor with reduced stray field effects. The compact design also improves the shielding of the sensitive electrodes and may also be used as an electrode cartridge without needing additional housing as the sensitive area can be internally sealed. In some embodiments, the sensitive area is internally sealed by soldering, and can withstand applied pressure from a material sensor without requiring an additional housing.

Further, a stacked sensor can utilize smaller electrodes, allowing for the sensor stack to be integrated into an active or passive mixing nozzle at the material inputs as well as the material output. In a sensor stack, only one electrode with an edge connector may be configured to connect to lead wires. As illustrated in FIGS. 16A-16B, stacked sensors may include a temperature sensor and may include an elongated portion for embodiments where a stir stick is an appropriate vehicle for detecting conductivity. However, it is expressly contemplated that a stacked sensor of either configuration can, without the elongated portion, be suitable for placement in a conduit, such as that illustrated in FIGS. 7A-7D.

In some embodiments, a number of electrode slots may be organized in a row, such that each slot is roughly the same distance from a connection end of the sensor (e.g. the end that interacts with a signal reader directly, through a wired system, or wirelessly. It is also illustrated herein that a number of electrode slots may be organized in a column, such that each slot has a different distance from the connection end. It is also expressly contemplated that, in some embodiments, electrode slots may be organized in both rows and columns. Sensors may provide desirable features of both sensor configurations of FIGS. 11A-11B and FIGS. 13A-13C.

A sensing setup, with a sensor partially submerged in a solution may include electrode slots of a first size and a second size. Electrode slots are arranged in both rows and columns. Arranging electrode slots in both rows and columns provides additional insight into a material.

The sensor may be used with a solution that is homogeneous or a solution that has experienced settling, which may be a sign of material age. Sensor may provide twelve different sensor signals for analysis, one from each electrode pair through which material can flow. A difference between signals from electrode slots may indicate aging. A difference between a signals from electrode slots may indicate a viscosity of solution.

Many production sites store raw material in large containers, such as drums. Material separation results in a lighter phase on top and a heavier phase on the bottom. Quality may decrease as separation increases.

Additionally, it is desired to have a sensor that can handle a wider viscosity range of materials. Electrode slots with smaller widths may not handle higher viscosity materials well, while electrode slots with wider widths may not be as precise for low-viscosity materials. Sensors may handle a wider range of viscosities while also providing signals along a depth of a material container. While only four rows of electrode pairs are illustrated, it is expressly contemplated that more rows may be present in other embodiments, to suit a container depth. Additionally, while only three columns are illustrated, it is expressly contemplated that additional columns with wider or narrower electrode slots are also possible.

Signal profiles that may be received from a sensor may indicate a low viscosity material while the other sensor indicates a high viscosity material.

Sensor systems herein may be used to detect a viscosity of a material in the comparison between profiles. The viscosity of a material impacts the exchange rate of material within a gap between electrodes. The delay between the signals of the large gaps and the signals of the smaller gaps indicates the viscosity of the material. A lower viscosity results in a shorter delay while a higher viscosity results in a longer delay.

It is expressly contemplated that sensitivity may be increased by stacking sensors as described for example in FIGS. 16A-16B.

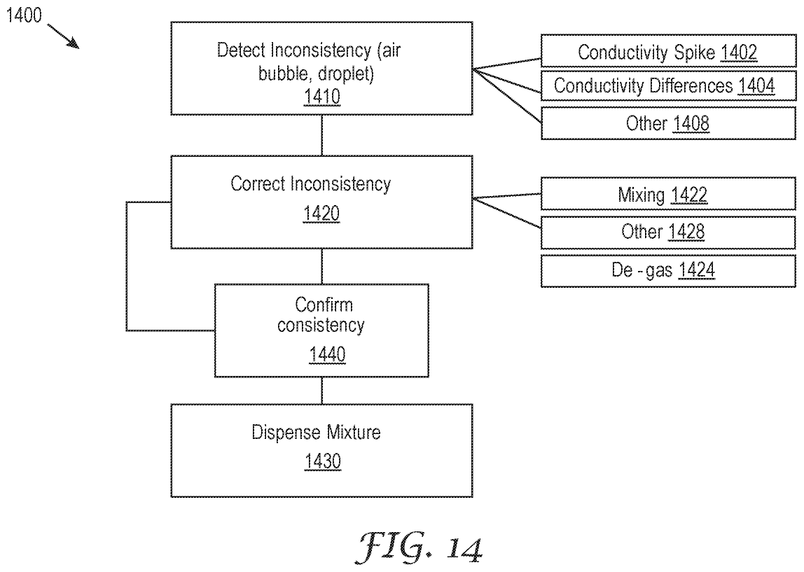

FIG. 14 illustrates a method for detecting and correcting quality concerns in a mixture in accordance with embodiments herein. Method 1400 may be practiced using sensor systems such as those described herein, combinations thereof, or other suitable sensors.

In block 1410, an inconsistency in a mixture is directed. The inconsistency may be entrained air, a mix ratio drift, inconsistent mixing—droplet formation, sedimentation, creaming—or another inconsistency from normal flow. Detection may be accomplished by detecting a spike, as illustrated in block 1402, in a sensed electrical parameter by one or more electrode pairs on a PCB sensor. Detection may also be accomplished by detecting a variation in sensed values, as illustrated in block 1404, measured between a first electrode pair and a second electrode pair of a sensor system. Other detection methods 1408 described herein may be used. Detection may occurs as a mixture flows through, or past, an electrode pair.

The electrical parameter sensor may be a disposable sensor intended to be discarded after use, in some embodiments. The sensor may include one or more pairs of electrodes in a coplanar arrangement such that the dispensed material flows through different electrode pairs. The sensor may also, or alternatively, include multiple sensing areas in an in-line arrangement such that material flow is parallel, or substantially parallel to, the sensing area. The inclusion of multiple electrode pairs of electrodes of varying sides may help to detect air bubbles or droplets of varying sizes as they flow through a sensing area.

In block 1420, the detected inconsistency is corrected. Correction may include further mixing 1422 the mixture, for example to ensure a consistent concentration, correct a detected mix ratio drift, reduce the risk of phase separation, and/or stabilize a dispersion or emulsion. Correction may also include degassing the mixture 1424, either to remove a detected air bubble or to remove entrained air introduced during a remixing step. Degassing may be accomplished using a vacuum, for example, or by purging a portion of the mixture containing the entrained air. Other suitable correction measures 1428, such as correcting a mixture composition, may also be used, such as a purge.

In some embodiments, it may be possible to mitigate a detected air bubble without purging, for example by instead sending a signal to the motor controlling fluid flow to increase speed and dispense an amount of material needed to replace the volume of air occupied by the bubble. In some embodiments, an applied pressure may increase, or a volumetric flow rate increased, in order to provide a similar volume of material if the bubble was not present.

In block 1440, consistency of the mixture may be confirmed prior to dispensing the mixture, in block 1430. For example, using sensors described herein, the consistency of the mixture may be confirmed by conductivity spikes stabilizing, e.g. reducing in severity and/or number, or by confirming that conductivity differences in electrode pairs have narrowed to an acceptable level. If consistency is not confirmed, the process may proceed back to block 1420 so that correction can be continued, or a new correction strategy may be selected.

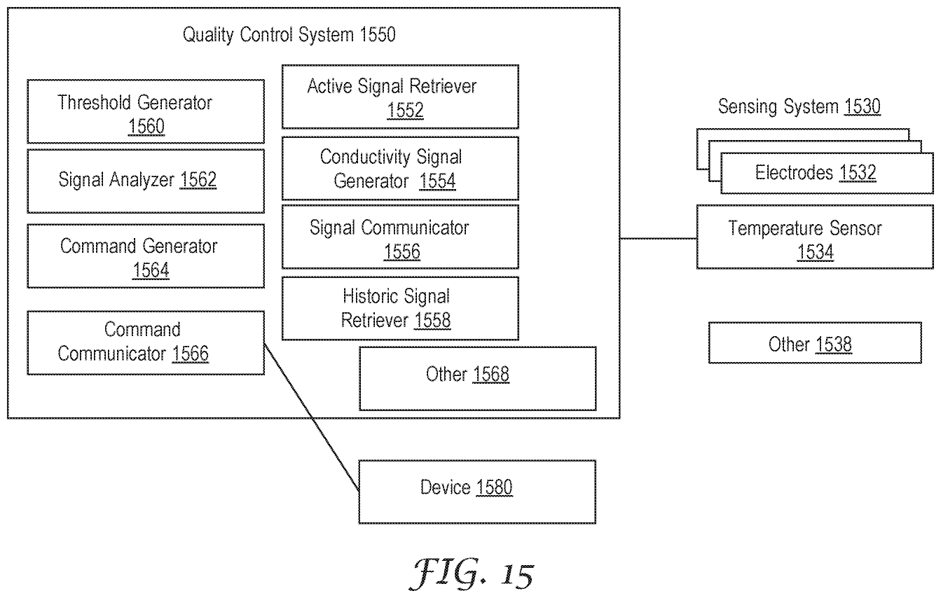

FIG. 15 illustrates a quality control system, which may also be described as an inconsistency detection system, in accordance with embodiments herein. Quality control system 1550 may be used to identify and correct a detected inconsistency in a mixture. Quality control system 1550 may be implemented in a static environment—e.g. as a dip stick or other analysis tool for a contained fluid—or a dynamic environment—e.g. in a fluid flow conduit where fluid moves through electrode pairs in a PCB board.

Some systems and methods herein may benefit from using relative thresholds instead of absolute thresholds. Base levels may be important to measure to have a more accurate relative threshold. For example, if a conductivity measurement drops below a proportionate factor to the base level (e.g. to 50% of the base level) then it can be determined that an inconsistency is present—e.g. a concentration gradient indicative of poor mixing, droplets indicative of phase separation, or entrained air. Relative thresholds may be helpful to reduce waste of material on accidental purges, or wasted time in attempting to correct an inconsistency that may not be present, or may not be at a level that requires correction.

Inconsistency detection system 1550 may be implemented by a suitable computing device in communication with a sensing system 1530. Sensing system 1530 may include one or more electrode pairs 1532 in direct contact with a material flow. Electrode pairs 1532 may be positioned such that fluid flows between them, or such that fluid contacts a surface of them. Electrode pairs 1532 may be part of a printed circuit board, for example, formed within apertures machined or built into the printed circuit board. The apertures may be closed on both ends, or open on one end, in a comb-like structure, for example. Electrode pairs 1532 may be printed onto a PCB. Printed electrode pairs 1532 may be arranged in a comb-like structure. Sensing system 1530 may also include a temperature sensor 1534. Temperature sensor 1534 may be shielded from direct contact with a material flow, in some embodiments. Sensing system 1530 may include other features 1538.

Sensor signals from sensing system 1530 are received by quality control system 1550 using an active signal retriever 1552. Active signal retriever 1552 may receive signals from sensing system 1530 periodically or continuously during an operation. Received sensor signals may be impedance signals, conductivity signals, dielectric constant signals, or a combination thereof. In embodiments where a conductivity value is used to detect an inconsistency, a conductivity signal generator 1554 may convert a received signal to a conductivity value. The signal value, and/or the conductivity value, may be provided to a data store, for example using signal communicator 1556. A similar process may be done for applications where a different electrical parameter is preferred for analysis purposes.

A historic signal retriever 1558 may communicate with a data store to retrieve previously captured signal values. Historic signal values of interest may include signal values retrieved in a recent period of time, from the same batch or mixture of materials. For example, values retrieved over a previous number of seconds or minutes may be important. In some embodiments, signal values may drift over longer periods of time due to changes in temperature, material aging, mixture ratio fluctuations, etc. But inconsistencies may be detectable as a rapid change in conductivity or a divergence of conductivity measurements in a sensing system from each other. Threshold generator 1560, in some embodiments, generates a relative threshold either periodically or continuously, based on historic signals. The relative threshold may be an absolute value, for example specifying that an increase or decrease of X % over Y time indicates an inconsistency. If conductivity values have fluctuated more significantly, the threshold change value may be larger, while if conductivity values have not fluctuated significantly, the threshold change value may be smaller.

Signal analyzer 1562 compares the received signal, or calculated conductivity, to the threshold and, if a deviation outside the allowed threshold is detected, command generator 1564 generates a command, which is communicated, using command communicator 1566, to a device 1580.

Device 1580 may, in some embodiments, include a display component, and the generated command may be an update to a graphical user interface, presented on the display component, indicating the detected inconsistency. Device 1580 may, in some embodiments, include a feedback component, such as audio, visual or haptic feedback that indicates to a controller that an air bubble is detected. Device 1580 may also be a correction mechanism, and command generator 1564 may generate a command to conduct a correction mechanism selected based on the detected inconsistency, e.g. a purge valve, a re-mixing command, a degassing command, etc.

System 1550 ay include other features 1568.

In some embodiments, threshold generator includes a machine learning model to forecast the conductivity time series data into the future from historical data. This forecast may include a so-called confidence intervals. The training may be done upfront on a reference data set with no detected quality control concerns, or with quantified quality control concerns. Signal analyzer 1562 then compares a received signal to determine whether it falls within, or outside of, the confidence interval.

In some embodiments, at regular intervals (e.g., 10 ms, 100 ms, etc.), threshold generator generates a prediction for the conductivity value, with confidence bands based on the historic signals retrieved by historic signal retriever. If the actual value measured drops below a lower confidence band, or goes above a higher confidence band, signal analyzer detects an inconsistency. If the conductivity measurement is within the confidence bands, signal analyzer 1562 provides an output that no inconsistency, or no inconsistency requiring correction has been detected. Command generator 1564 may provide an indication that a GUI of device 1580 does not require updating.

A relative threshold is an important component of an air detection system because of the noise present in the data. The statistical concept of confidence bands can account for this-if data have more noise, the confidence bands are further away from the current value and the inconsistency detection algorithm will not yield wrong detections just because of noisy data, where a simple thresholding approach can suffer from this in this case.

While conductivity is discussed herein as the value of interest, it is expressly contemplated that other material parameters, such as the amount of electrical current, the relative permittivity (er) or impedance could be used instead or as well for the detections algorithm.

Measuring conductivity can provide valuable information regarding quality of a mixture. For example, as described herein, and in the Examples Section of PCT/US2022/052343, conductivity measurements may be used for determining consistency issues due to lot-to-lot variation, entrained air, droplet formation, aging, concentration gradients, dispersion separation or emulsion separation.

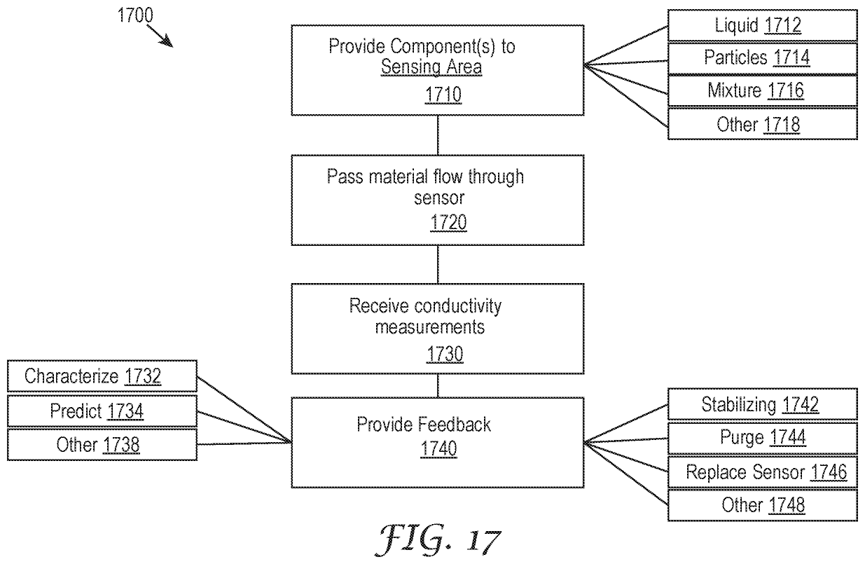

FIG. 17 illustrates a method of quality controlling a material dispensing system in accordance with embodiments herein. Method 1700 may be used with the dispensers described herein, or another suitable sensing system.

In block 1710, one or more components to be dispensed are provided to a sensing area. The sensing area may be a material dispenser, a transport line to a material dispenser, before a nozzle, atomizer, or other transportation mechanism or container within a fluid system. For example, a material dispenser may dispense a liquid 1712, particles 1714 either in suspension or otherwise. The material may also be a mixture 1716 of materials, for example an emulsion or another A and B component mixture. An emulsion must be dispensed as a stable emulsion, and reactive A: B components should be provided at a desired mix ratio. Other components 1718 may also be provided to a sensing area prior to dispensing.

In block 1720, the mixture passes through a sensing system before, for example before being dispensed, stored, removed from storage. Passing through a sensing system may entail passing through a portion of a sensing body such that the material (e.g. a mixture or a component) directly contacts a sensor. Direct contact between a material and an electrode pair ensures accurate measurements. Passing through a sensing

In block 1730, conductivity measurements are received from the sensing system. The sensing system may have multiple sensors, for example a plurality of electrode pairs that, when a sufficient voltage is passed through them, detects an electric parameter of the material. Based on the sensed parameter value, a number of things may be determined for the material. For a mixture, a mixing ratio may be determined. For a curable material, a curing progressing may be detected. Aging may also be detectable, as well as differences between batches of materials. Instability indications—such as entrained air, impending phase separation, etc. may also be detectable. Sensor measurements may be taken serially, for example one signal received every second, or more frequently. Measurements may also be taken in parallel, for example from each of a plurality of electrode pairs or sensing areas. The electrode pairs or sensing areas may be coplanar with each other, in some embodiments. Electrical parameters sensed may include conductivity, impedance or dielectric constant or another suitable parameter.

In block 1740, feedback is provided based on the electrical parameter measurements. Feedback may include characterization of the material, as indicated in block 1732. For example, a mix ratio may be detected, entrained air or single component fluid pockets, an age indication or other parameter of interest may be calculated and provided. A prediction may also be provided, as indicated in block 1734. For example, based on a trend of previous conductivity sensor readings, it may be possible to predict future behavior of the material being measured. Other characterization information 1738 may also be provided. For example, a conductivity reading trending in one direction may indicate that a mix ratio is moving toward an edge of an acceptable range and, therefore, that a mix rate should be changed, or that an increase in instability is trending toward phase separation. Similarly, a conductivity reading may indicate that a curable component is curing.

Feedback may also indicate corrective action is needed. For example, an emulsion or dispersion experiencing separation may need stabilizing 1742—e.g. remixing, heating, etc. Feedback may also indicate that a purge of one component, multiple components, or a mixture, is needed, as indicated in block 1744. In embodiments where a material has corrosive effects, or cures over time, predictive feedback may provide an indication that the sensor needs to be replaced, as indicated in block 1746. Other predictive information may also be provided that may trigger other actions, as indicated in block 1748.

In some embodiments, as illustrated herein, providing feedback may also include providing conductivity readings, material characterizations or predictions to a customer, controller of a dispenser, or other useful information such as material source, batch number, material name, dispensing temperature, dispensing pressure, material concentration(s), mix ratio, or any other information.

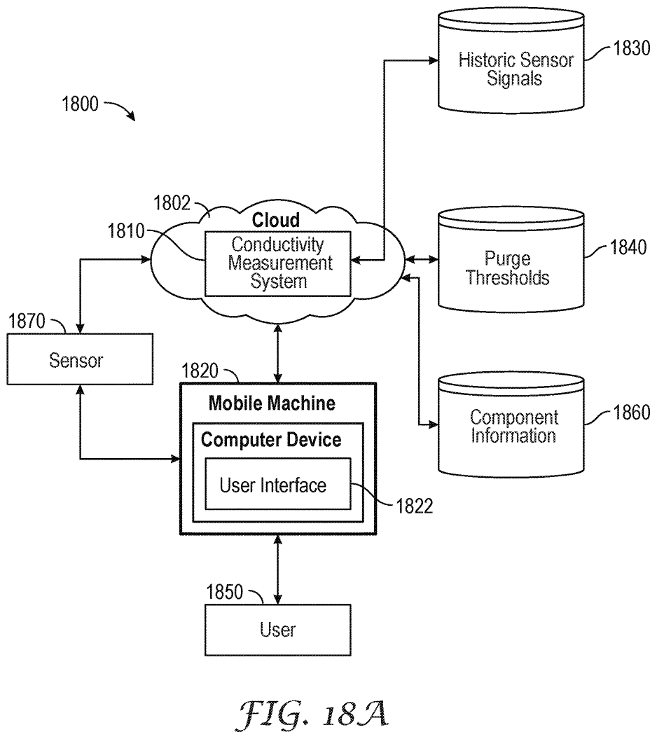

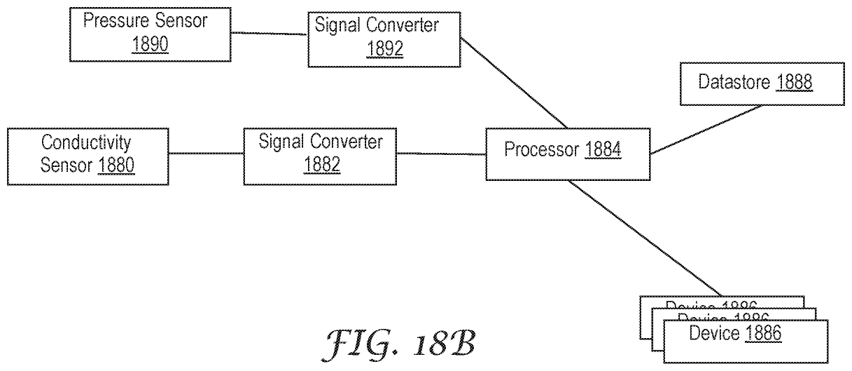

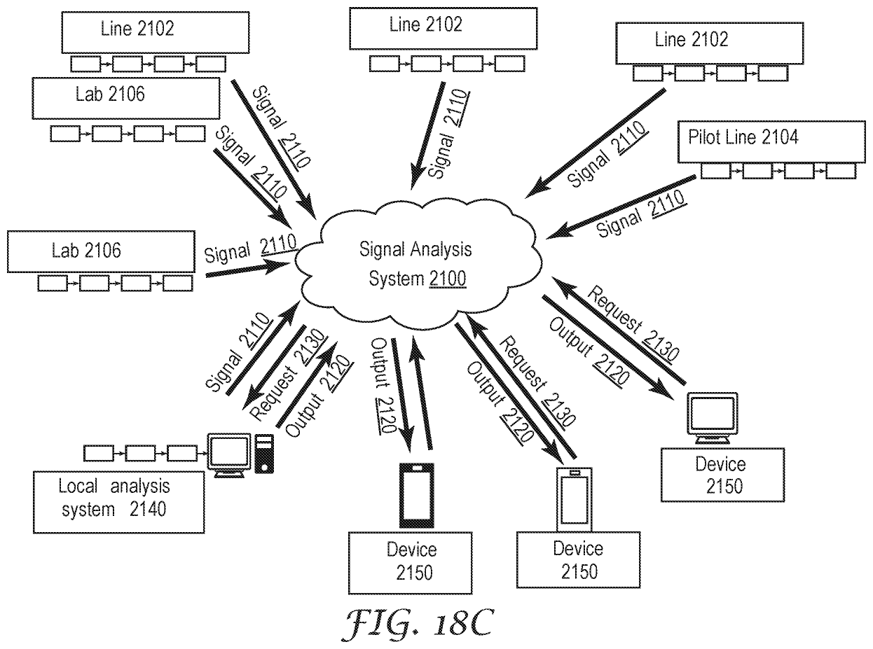

FIGS. 18A-C illustrate a conductivity measurement system in a network of systems in accordance with embodiments herein.

In the example shown in FIG. 18A, some items are similar to those shown in earlier figures. FIG. 18A specifically shows that a conductivity sensing system 1810 can be located at a remote server location 1802. Therefore, computing device 1820 accesses those systems through remote server location 1802. User 1850 can use computing device 1820 to access user interfaces 1822 as well. For example, a user 1850 may interact with an application on the user interface 1822 of their smartphone, or laptop, or other computing device 2320, which may also be described as a mobile machine or computer device, to receive information from a dispensing system or a quality control system.

FIG. 18A shows that it is also contemplated that some elements of systems described herein are disposed at remote server location 1802 while others are not. By way of example, data stores 1830, 1840 and/or 1860 can be disposed at a location separate from location 1802 and accessed through the remote server at location 1802. Regardless of where they are located, they can be accessed directly by computing device 1820, through a network (either a wide area network or a local area network), hosted at a remote site by a service, provided as a service, or accessed by a connection service that resides in a remote location. Also, the data can be stored in substantially any location and intermittently accessed by, or forwarded to, interested parties. For instance, physical carriers can be used instead of, or in addition to, electromagnetic wave carriers. This may allow a user 1850 to interact with system 1810 through their computing device 1820, to initiate a seal check process.

It will also be noted that the elements of systems described herein, or portions of them, can be disposed on a wide variety of different devices. Some of those devices include servers, desktop computers, laptop computers, imbedded computer, industrial controllers, tablet computers, or other mobile devices, such as palm top computers, cell phones, smart phones, multimedia players, personal digital assistants, etc.

A conductivity measurement system may be any suitable system configured to, using systems and methods herein, collect conductivity measurements, conduct analysis and provide the analysis to a receiving device, storage or graphical user interface generator. FIG. 16 of PCT/US2022/052343, and related description, discloses operation of such a system and is hereby incorporated by reference.

System 1810 receives conductivity measurements from one or more sensors 1870. Each sensor may include one or more pairs of electrodes on a PCB. The electrodes may be coplanar and spaced similarly away from one end of the PCB, in some embodiments, or may be coplanar and in line with a length of the PCB. Sensors may be formed either by metallization or another process. Sensors 1870 are decoupled from each other such that independent conductivity signals are received from each sensor. Sensors 1870 may each include a positive and negative electrode, decoupled from one another.

Conductivity measurement systems 1810 may receive a sensor signal as a conductivity signal or a dielectric constant signal, or an impedance signal. In embodiments where a received signal is an impedance signal, a conductivity value may be calculated based on the impedance signal. similarly, a dielectric constant may be calculated based on a received impedance signal. Based on received sensor signals, calculations and/or predictions may be undertaken, as described herein. A mixing ratio may be calculated based on calibration data, stored in a datastore 1860, which may be indicative of conductivity data from pure components and/or known mixtures of components. As described above, sensors may be placed at both the inlets and outlet of a sensing zone and, therefore, system 1810 may receive sensor signals from all sensors associated with a material dispensing system. System 1810 may be configured to correct for the time delay between sensor signal capture and analysis, in some embodiments. In other embodiments, where trend information is particularly relevant, correction may not be needed.

Systems and methods are described herein that take advantage of machine learning algorithms. Machine learning models may be preferred because they can better handle noisy data, make predictions about future signal trends, and make adjustments before mix quality significantly shifts. Systems and methods described herein can calculate the mix ratio real-time. With machine learning techniques, the mix ratio could be predicted ahead of time. This allows quicker adjustments which keeps the mix ratio closer to the target value more of the time. With some current dispensers, a lot of material is entrained in the static mixer, such that, by the time a shift in mix ratio is detected, the material already in the mixer will continue to have the wrong mix ratio for at least a mixer's worth of adhesive, so identifying mix ratio issues earlier can save material and a potential purge.

Similarly, machine learning models, as described herein, may receive information from multiple systems, such as multiple sensors within a dispensing system including conductivity sensors, temperature sensors, motor speed signals, material information, etc. In some embodiment, multiple machine learning models are used simultaneously, each by an individual system such that each system's model can learn and the overall model can be improved. However, it is also expressly contemplated that non-machine learning models may also be used.

Sensing systems herein are described as having the functionality of receiving and sending communicable information to and from other devices. This may be done through an application program interface, for example, such that system 1810 can receive and communicate with pump controllers, line pressure sensors, movement controllers for portions of dispensing system, temperature sensors, heating elements, datastores having information for any of the materials being dispensed or the mixture being generated, etc.

In embodiments where machine learning models are used, datastore may also include an analyzer that learns usage behavior of a particular dispensing system in order to improve operation and predictions. Similarly, frequency and patterns of dispensing may provide information about curing and improve mixing models. For example, usage data such as frequency of dispense, purging frequency, pattern of dispense, change out of the sensor, etc., can be collected and used to train a model to more accurately predict trends and provide corrective action.

Similarly, as described herein, user interface 1822 may display a GUI that is updated periodically with information collected by system 1810 and/or any of datastores 1830, 1840, 1860. Information may be passively updated or provided with an alert or notification as it is updated, for example current status information may be presented and an alert (visual, audio, or haptic) may be provided if the mixing ratio is drifting toward an unacceptable range. Additionally, or alternatively, notifications may be provided when a device command is generated, or when operator intervention is needed.

In some embodiments, a signal encoder and regressor may operate locally, for example using a computer processing device associated with a material dispensing system. Alternatively, either encoder or regressor, or both, may be deployed in a cloud-based storage system.

The output of encoder may be directly used to apply pressure changes on the cartridges associated with one or more material components to ensure that the mixture meets a predefined mixing ratio. E.g., if the mixed material contains too much of part A, the pressure on the cartridge containing part A is reduced and the pressure on the cartridge that contains part B increased.

A regressor may then take the encoded signals and produce a mixing ratio signal. The regressor may be a machine learning based algorithm that can be trained in any suitable way.

A first training option is a separate training option where the Encoder-Decoder model is trained on a set of signals of a variety of parts for part A, part B, and diverse mixtures. The Machine Learning Regressor is trained in a second step afterwards on the encoded signals and the corresponding mixing ratios.

A second training option is an alternating training option, where one batch of signals is used for one training step in the Encoder-Decoder and then used for one training step in the Encoder-Machine Learning Regressor part. A training step consists of a forward pass of the data in a batch, the calculation of the gradient, and an application of the gradient to optimize the weights in the model.

A third training option is a combined training option where the triplet of Encoder-Decoder pair and Machine Learning model are optimized simultaneously. This means that a batch is forward through the Encoder, and the representation obtained is forwarded through the Decoder and the Machine Learning Regressor. Then the gradients calculated with both outputs are applied in a weighted combination in the backwards pass.

Alternating or combined training may provide a benefit in that the representation of the signals is learned in a way that it has a positive effect on the performance of the Regressor which can lead to a lower error when estimating the mixing ratio. Learning a representation of signals on a variety of materials and mixing ratios also allows the models to be used on previously unseen materials of the same chemical family.

In difference to a system which only uses a single signal from the mixed material, this novel approach allows adaption for lot-to-lot variation of the raw material, where a change in one of the parts can lead to a change in the mixed signal for the same mixing ratio. It also enables tracking the mixing of the new materials of the same family be learning to fuse the signals of two parts into a mixed signal.

Data traces collected from a sensor system can be processed to provide other information as described herein. For example, sensors may provide signals that can be processed to indicate that corrective action is needed.

As described herein, in some embodiments, a sensor includes four electrode pairs. A time series of conductivity can be analyzed from the four sensor capacitors to determine when corrective action has been successful—e.g. when remixing has completed, when phase separation is reversed or a mixture has again reached stability.

For example, mixing (or remixing) may take time to reach a steady state. For example, when starting a mixing operation, backpressure and different viscosities of components can cause mixing to start off poorly and gradually stabilize. The same variance can be used to track the stabilization and indicate when the dispenser can dispense material on a workpiece or to a receiving container. The trend of the variance can be analyzed against a threshold. The threshold is specific for each material. However, instead of determining a threshold, the signal can be tested for stationarity using the Augmented Dickey-Fuller (ADF) test. The advantage with this is that manual thresholds often need to be tuned for a new batch, but the ADF test is adaptable.

Inhomogeneity can also be detected using sensors described herein. The four electrode pairs should also record similar readings. Some constant offset is possible due to manufacturing tolerances, but in a stable mixing process, the variations of the four signals should be synchronous.

Once each signal has stabilized, the four sensors should have a high covariance. Negative covariance indicates a persisting anti-correlated behavior and signifies spatial inhomogeneity.

Similarly, a single component of a mixture can also be inhomogeneous, e.g., because of settling in the barrel or insufficient mixing during manufacturing. An ADF test can again be used to confirm stationarity over a longer time. The relevant time frame would be determined by the time it takes to empty the container.

Architecture 1800 illustrates one embodiment of an implementation of a electrical parameter sensing system 1810. As an example, architecture 1800 can provide computation, software, data access, and storage services that do not require end-user knowledge of the physical location or configuration of the system that delivers the services. In various embodiments, remote servers can deliver the services over a wide area network, such as the internet, using appropriate protocols. For instance, remote servers can deliver applications over a wide area network and they can be accessed through a web browser or any other computing component. Software or components shown or described in the present disclosure as well as the corresponding data, can be stored on servers at a remote location. The computing resources in a remote server environment can be consolidated at a remote data center location or they can be dispersed. Remote server infrastructures can deliver services through shared data centers, even though they appear as a single point of access for the user. Thus, the components and functions described herein can be provided from a remote server at a remote location using a remote server architecture. Alternatively, they can be provided by a conventional server, installed on client devices directly, or in other ways.

FIG. 18B illustrates an example system architecture. In the embodiment of FIG. 18B, the system is connected through wires, such that it is not a wireless or open distributing solution. Wired communication may also be preferred in embodiments where a wireless connection would have slower transfer rates or potentially unreliability. However, as discussed with respect to FIG. 18A, it is contemplated that wireless systems may also be possible.

An electrical parameter sensor 1880, which may also be described as a conductivity sensor, may capture an electrical parameter signal, for example from one or more PCB sensors described herein, provide that sensor signal to a signal converter 1882 where, if needed, signal conversion occurs. However, it is expressly contemplated that in some embodiments sensor 1880 may provide a sensor signal directly to processor 1884. Signal converter 1882 may convert, for example, impedance to conductivity, an analog to a digital signal, or may do another suitable conversion.

Processor 1884 receives the electrical parameter indication, and generates an electrical parameter value output, which may be provided to one or more devices 1886. Devices 1886 may include a computing device with display, a smart phone with display, a laptop with display, or to another device, for example a storage medium which stores the sensor signal for future reference. Processor 1884 may also consult one or more data stores 1888 in order to generate additional indications. For example, data store 188 may include past conductivity sensor signals, conductivity sensor signal thresholds, commands to adjust dispensing parameters based on conductivity signal thresholds, etc. Processor 1884 may act accordingly.

In accordance with embodiments herein, system may also have a pressure sensor 1890 that generates a pressure signal, indicative of a detected pressure at a point within the dispensing system. If needed, a signal converter 1892 may convert the pressure signal from one form to another, from ampere to voltage, analog-to-digital, etc.

Processor 1884, or another suitable processor, may generate a pressure output, which may be provided to one or more devices 1886. Processor 1884 may receive signals from pressure sensor 1890 and conductivity sensor 1880 continuously throughout a process, and may be able to generate outputs continuously as well, providing substantially real-time information about a dispensing system. Processor 1884 may include one or more suitable machine learning techniques, may consult a lookup table, or perform another suitable data analysis technique on a received conductivity signal or pressure signal.

Processor 1884 may communicate with sensors 1880, 1890 wirelessly, using a wired connection, or through any other suitable network. Processor 1884 may receive signals as encrypted signals, may provide output as an encrypted output, or may operate without encryption protocols in place.

Any number of suitable communication routes are envisioned, e.g. from sensor 1890 directly to processor 1884, from sensor 1880 through signal converter 1882, and directly to datastore 1888, where it may be retrieved by processor 1884. Similarly, a request for information from devices 1886 may be sent directly to conductivity sensors 1880, 1890, to datastore 1888 or to processor 1884.

In some embodiments, an MQTT broker is used to allow, for example, devices 1884 to subscribe to a subset of data from sensor 1890 or processor 1884, for example.

In some embodiments, processor 1884 also communicates with data store 1888, such that conductivity and pressure signals are also stored for later analysis. For example, a data set including conductivity and pressure signals over time may be used to train a machine learning algorithm, or may be used for troubleshooting purposes. For example, a machine learning algorithm may be able to detect patterns in the data set, such as an off mix ratio and need to purge, and provide indications and or thresholds about how to detect when mix ratio deviation occurs before the deviation become severe.

FIG. 18B illustrates a single processor that receives information from a single set of sensors for a dispensing operation. However, it is expressly contemplated that a production environment may have multiple dispensers running with multiple conductivity sensors and pressure sensors providing status information continuously. It is anticipated, therefore, that multiple users may want to view information about multiple production lines at the same time. FIG. 18C illustrates one configuration of a system that may be able to provide such functionality.

FIG. 18C illustrates a signal analysis system that communicates with a number of devices using a cloud-based network. As illustrated in FIG. 18C signal analysis system 2100 may communicate with a local analysis system 2140, such as that described with respect to FIG. 18B. Signal analysis system 2100 may receive a number of sensor signal data 2110 from a number of dispensing operations, such as a pilot line 2104, any of an operational line 2102, and/or a laboratory set up 2106. As described with respect to FIG. 18B, sensor signals 2110 may be digital signals, analog signals, conductivity measurement signals, pressure signals, or other signal information. For example, a low reservoir detected signal, a valve switch indication, or any other detectable indication from any of systems 2102-2106.

Signal analysis system 2100 may conduct analysis on receive sensor signal information 2110, for example using any suitable analysis tool such as lookup table, comparison thresholds, and/or machine learning algorithms to detect parameter trend information that may indicate a problem, or an action that needs to be taken, such as purging, adjusting mix ratio, etc.

Signal analysis of 2110 may provide output indicia 2120 a number of suitable devices 2150. Signal analysis system 2100 may provide output information 2120 continuously, or in response to a request 2130 information. The request 2130 may be a one-time request for current status information, or a request to receive continuous updates going forward.

Current sensing setups include components from different manufacturers, and data preparation and processing is done using a separate computing device. However, it is desired to have a robust and compact system that processes data quickly with limited downtime or booting time, such that quality concerns with a material are detected quickly.

In some embodiments herein a sensor contains signal preparation and processing within a single housing, e.g. a “smart” sensor. Such smart sensors contain a processing component—e.g. a microprocessor, a microcontroller, a digital signal processor or other processing circuitry. In some embodiments, a sensor also includes one or more standardized interfaces for interfacing with other systems—e.g. fieldbus systems, sensor networks, input/output links, etc. In some embodiments herein, sensor signal processing is completed without an external computer. Sensing systems herein provide decentralization, increased reliability, reduced cost, increased flexibility and simplification.

In some embodiments, a sensor system herein includes a concentrator which integrates electronic parts in a single housing. In some embodiments, all electronic components are on one PCB. In some embodiments, an analog frontend with signal conversion (e.g. AD-Converters, DA-Converters or both) are connected to a microcontroller that performs signal converting, processing and provide an output signal. Sensing systems herein may also incorporate operational circuitry, including power-supply, I/O protection circuitry, signal conditioning, reset management and/or debugging circuitry and interfaces. In some embodiments herein, the concentrator includes user-interface components such as LED signaling, UART, USB, wireless interfaces (e.g. Bluetooth®, WiFi, Zigbee®, cellular network), dot-matrix or alphanumeric display, industrial bus systems and/or tactile interface components such as push-buttons, switches, touchscreens, etc.

Systems herein may include user accessible data—e.g. a signal value, a pass/fail (e.g. “yes” or “no,” “go” or “stop,” etc.). Systems herein may provide a quality or quantity indication. Systems herein may provide a data stream with time and/or frequency-dependent data for storage and/or further processing. Systems herein may include algorithms and/or calibrations needed for data manipulation.

In some embodiments, a sensing system may be used with sensor described in embodiments herein, for example, or with another suitable sensor. A sensor signal reader connects to a sensor, for example an edge connector of a PCB-board that includes one or more electrode pairs. In some embodiments, a trans-impedance amplifier is present to convert current measurements to voltage. A concentrator receives sensor signals, processes said sensor signals, and provides an output. An output may be provided using an I/O device and/or another wired or wireless communication protocol. A power source may provide power to concentrator. It is possible that power may be provided wirelessly, or concentrator may be integrated into a material dispensing system from which it draws power.

One example interface of a concentrator may receive sensor signals using one or more sensor signal receiving ports. Other data or inputs may be received through another receiver, in some embodiments.

Another interface may receive a coupling to an input/output device. Power may be provided, for example using a port. Data may be communicated from a concentrator using a computer link.

Components of a sensing system in accordance with embodiments herein may include one or more sensors 2542 provide sensor signals, received by one or more receivers coupled to, or included within, a housing. In some embodiments, the system includes an analog front-end which may include a filter and/or an analog multiplexor. A converter, e.g. a DA- or DC-converter may be present. Concentrator may include non-volatile memory, flash memory, or another suitable information storage. A temperature sensor may be incorporated into concentrator, or receive a temperature signal from a temperature sensor. Concentrator may include a clock. Concentrator may also include reset functionality.

Multiplexor may be of particular importance in embodiments where a larger number of sensor signals are received—for example from more than a few sensing areas. In some embodiments, multiplexor may facilitate switching between receiving signals from a first plurality of sensing areas to a second plurality of second sensing areas, etc. Described herein are embodiments where sensor signal scan be retrieved from four independent sensing areas. However, it is expressly contemplated that a greater number of sensing areas may be present for some applications. For example, a fluid or mixture may be stored in a large drum. To determine whether separation is occurring, it may be helpful to have sensing areas along a portion, or the entirety, of the height of the drum. Up to 80, or more, sensing areas may be required to cover that distance. Multiplexor may switch between groups of sensing areas in order to obtain an understanding of the state of the mixture throughout the entirety of the storage container.

A sensor analyzer may include calibration data and/or functionality. A real-time operating system may manage functionality. Sensor analyzer my include Fourier transformer. Sensor analyzer may include a waveform generator. Sensor analyzer may include other applications that provide other functionality, such as detecting of material characteristics like mix ratio, material age, curing progress, etc. Sensor analyzer may also include an identifier that identifies a type of sensor.

Concentrator may include a power management system that includes, or accesses, a power supply. A power quality may be monitored. Energy consumption may be tracked. Conversion input and output ranges may be stored. A symmetric voltage may be used.

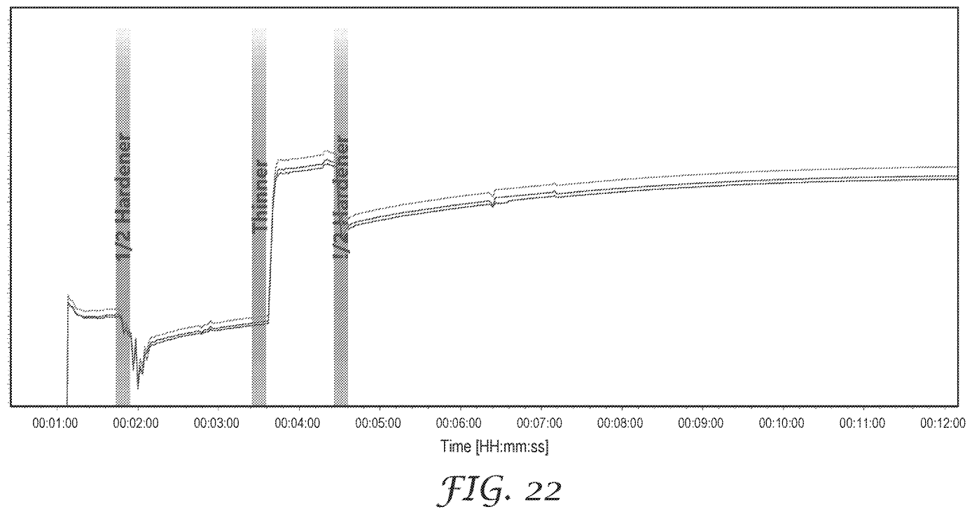

FIG. 22 illustrates a dispensing system in accordance with embodiments herein. Many dispensing operations are done with a portable, handheld system. Errors in dispensing or adhesive failure can result if material quality or machine settings are not correct. For example, an incorrect mix ratio or an incorrect pressure setting may result in an unacceptable product. It is desired to have a handheld dispensing system that can provide real-time sensing and feedback to a user. Described in FIG. 22 is one example of a system that can receive and process sensor signals without a separate computing device. Described herein are many embodiments of sensors that may be used with a dispenser. Described herein are systems for measuring pressure in a dispensing system. System 2600 includes a dispenser 2610. Dispenser 2610 is illustrated as an adhesive dispenser 2610, however other dispensers may also benefit from systems described herein. Dispenser 2610 includes an in-line sensor 2630 that senses electrical properties of a material being dispensed. A pressure sensor 2640 is incorporated into dispenser 2610 and monitors the pressure within the dispenser.

Dispenser 2610 also includes a signal processing system 2620. A signal receiver receives a sensed parameter signal from sensor 2630. A processing unit, which may include any suitable processor or processing circuitry, processes the sensed signal. A memory may store calibration data, historic signals, etc. A display 2650 may present processed information to a user, the information received from signal processing system 2620, for example using a communication module. Display 2650 may be integrated into dispenser 2610, or another display visible to a dispenser operator, such as a mobile computer, a worksite display, etc. However, while a display 2650 is illustrated as conveying processed information to an operator, it is expressly contemplated that output from signal processing system 2620 can be presented as audio or haptic feedback in some embodiments herein.

Based on sensed signals, signal processing system 2620 may also actuate a change in dispensing parameters. For example, a mix ratio may be sensed that as drifted away from a specified mix ratio. Signal processing system 2620 may, based on the sensed mix ratio drift, adjust a mix ratio by changing a pump speed for one component. Signal processing system 2620 may control pump speed directly, or indirectly, such that an instruction to change the pump speed is sent to a pump controller. Signal processing system 2620 may also communicate the mix ratio drift, e.g. through display 2650. In some embodiments, signal processing system 2620 may only communicate a detected material issue—e.g. mix ratio, aging, curing, pressure, etc.—and an operator may need to take steps to address the issue manually. However, it is expressly contemplated that, in some embodiments, dispenser parameters are adjusted automatically, in real-time, based on signals from sensors 2630, 2640.

Information about expected process parameters—e.g. mix ratio, dispensing pressure—may be detected in any suitable manner. In some embodiments, a dispenser receives expected process parameters from an NFC tag, RFID tag, or other information storage system on a material to be dispensed.

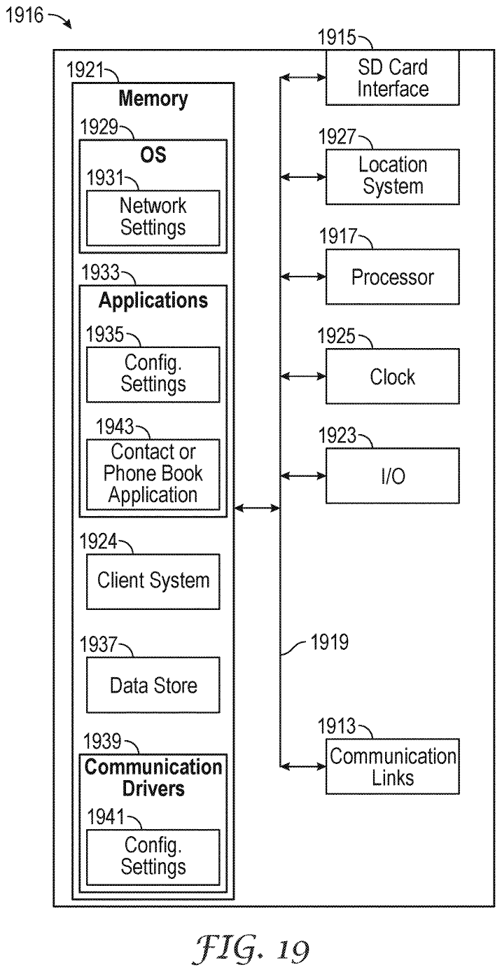

FIGS. 19-21 illustrate example devices that can be used in the embodiments shown in previous Figures. FIG. 19 illustrates an example mobile device that can be used in the embodiments shown in previous Figures. FIG. 19 is a simplified block diagram of one illustrative example of a handheld or mobile computing device that can be used as either a worker's device or a supervisor/safety officer device, for example, in which the present system (or parts of it) can be deployed. For instance, a mobile device can be deployed in the operator compartment of computing device for use in generating, processing, or displaying the data.

FIG. 19 provides a general block diagram of the components of a mobile cellular device 1916 that can run some components shown and described herein. Mobile cellular device 1916 interacts with them or runs some and interacts with some. In the device 1916, a communications link 1913 is provided that allows the handheld device to communicate with other computing devices and under some embodiments provides a channel for receiving information automatically, such as by scanning. Examples of communications link 1913 include allowing communication though one or more communication protocols, such as wireless services used to provide cellular access to a network, as well as protocols that provide local wireless connections to networks.

In other examples, applications can be received on a removable Secure Digital (SD) card that is connected to an interface 1915. Interface 1915 and communication links 1913 communicate with a processor 1917 (which can also embody a processor) along a bus 2719 that is also connected to memory 1921 and input/output (I/O) components 1923, as well as clock 1925 and location system 1927.

I/O components 1923, in one embodiment, are provided to facilitate input and output operations and the device 1916 can include input components such as buttons, touch sensors, optical sensors, microphones, touch screens, proximity sensors, accelerometers, orientation sensors and output components such as a display device, a speaker, and or a printer port. Other I/O components 1923 can be used as well.

Clock 1925 illustratively comprises a real-time clock component that outputs a time and date. It can also provide timing functions for processor 1917.

Illustratively, location system 1927 includes a component that outputs a current geographical location of device 1916. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 1921 stores operating system 1929, network settings 1931, applications 1933, application configuration settings 1935, data store 1937, communication drivers 1939, and communication configuration settings 1941. Memory 1921 can include all types of tangible volatile and non-volatile computer-readable memory devices. It can also include computer storage media (described below). Memory 2721 stores computer readable instructions that, when executed by processor 1917, cause the processor to perform computer-implemented steps or functions according to the instructions. Processor 1917 can be activated by other components to facilitate their functionality as well. It is expressly contemplated that, while a physical memory store 1721 is illustrated as part of a device, that cloud computing options, where some data and/or processing is done using a remote service, are available.

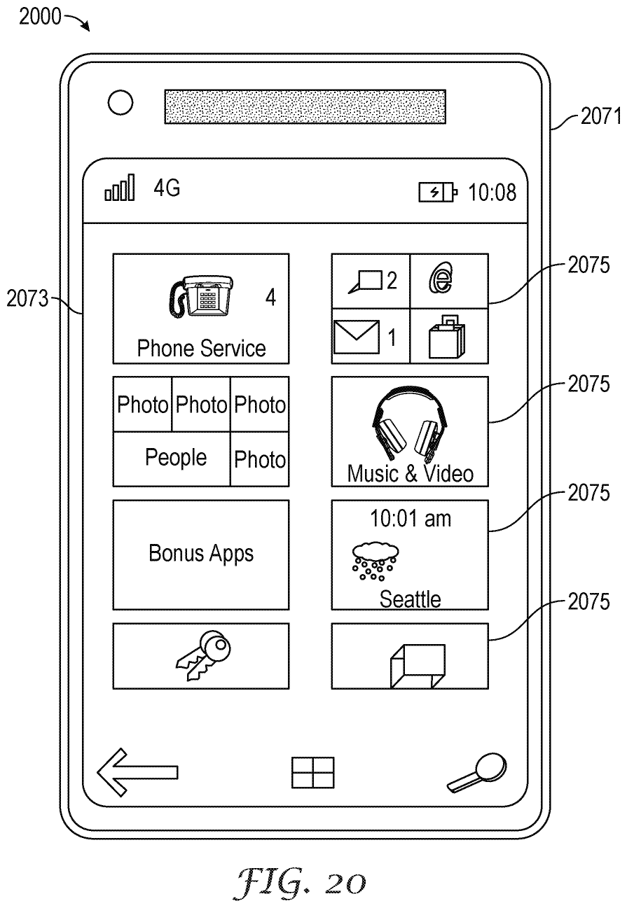

FIG. 20 shows that the device 2000 can also be a smart phone 2071. Smart phone 2071 has a touch sensitive display 2073 that displays icons or tiles or other user input mechanisms 2075. Mechanisms 2075 can be used by a user to run applications, make calls, perform data transfer operations, etc. In general, smart phone 2071 is built on a mobile operating system and offers more advanced computing capability and connectivity than a feature phone. Note that other forms of the devices are possible.

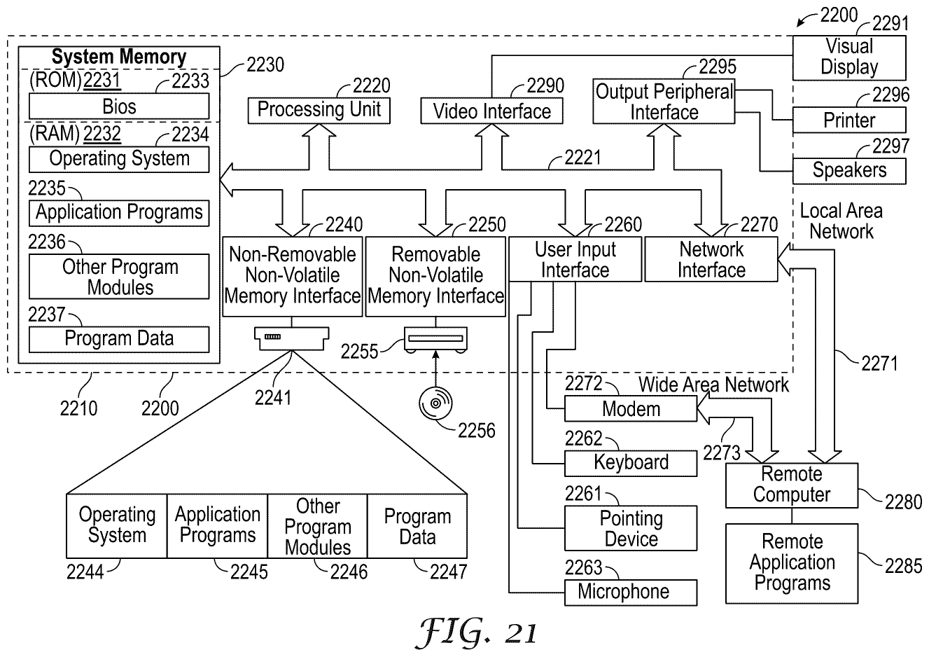

However, while FIG. 21 illustrates an embodiment where a device 2000 is a smart phone 2071, it is expressly contemplated that a display may be presented on another comping device.

FIG. 21 is one example of a computing environment 2200 in which elements of systems and methods described herein, or parts of them (for example), can be deployed. With reference to FIG. 21, an example system for implementing some embodiments includes a general-purpose computing device in the form of a computer 2210. Components of computer 2210 may include, but are not limited to, a processing unit 2220 (which can comprise a processor), a system memory 2230, and a system bus 2221 that couples various system components including the system memory to the processing unit 2220. The system bus 2221 may be any of several types of bus structures including a memory bus or memory controller, a peripheral bus, and a local bus using any of a variety of bus architectures. Memory and programs described with respect to systems and methods described herein can be deployed in corresponding portions of FIG. 21.

Computer 2210 typically includes a variety of computer readable media. Computer readable media can be any available media that can be accessed by computer 2210 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 2910. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 2230 includes computer storage media in the form of volatile and/or nonvolatile memory such as read only memory (ROM) 2231 and random-access memory (RAM) 2232. A basic input/output system 2233 (BIOS) containing the basic routines that help to transfer information between elements within computer 22210, such as during start-up, is typically stored in ROM 2231. RAM 2232 typically contains data and/or program modules that are immediately accessible to and/or presently being operated on by processing unit 2220. By way of example, and not limitation, FIG. 21 illustrates operating system 2234, application programs 2235, other program modules 2236, and program data 2237.

The computer 2210 may also include other removable/non-removable and volatile/nonvolatile computer storage media. By way of example only, FIG. 21 illustrates a hard disk drive 2241 that reads from or writes to non-removable, nonvolatile magnetic media, nonvolatile magnetic disk, an optical disk drive 2255, and nonvolatile optical disk 2256. The hard disk drive 2241 is typically connected to the system bus 2221 through a non-removable memory interface such as interface 2240, and optical disk drive 2255 are typically connected to the system bus 2221 by a removable memory interface, such as interface 2250.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 21, provide storage of computer readable instructions, data structures, program modules and other data for the computer 2210. In FIG. 21, for example, hard disk drive 2241 is illustrated as storing operating system 2244, application programs 2245, other program modules 2246, and program data 2247. Note that these components can either be the same as or different from operating system 2234, application programs 2235, other program modules 2236, and program data 2237.

A user may enter commands and information into the computer 2210 through input devices such as a keyboard 2262, a microphone 2263, and a pointing device 2261, such as a mouse, trackball or touch pad. Other input devices (not shown) may include a joystick, game pad, satellite receiver, scanner, or the like. These and other input devices are often connected to the processing unit 2220 through a user input interface 2260 that is coupled to the system bus but may be connected by other interface and bus structures. A visual display 2291 or other type of display device is also connected to the system bus 2221 via an interface, such as a video interface 2290. In addition to the monitor, computers may also include other peripheral output devices such as speakers 2297 and printer 2296, which may be connected through an output peripheral interface 2295.

The computer 2210 is operated in a networked environment using logical connections, such as a Local Area Network (LAN) or Wide Area Network (WAN) to one or more remote computers, such as a remote computer 2280.

When used in a LAN networking environment, the computer 2210 is connected to the LAN 2271 through a network interface or adapter 2270. When used in a WAN networking environment, the computer 22210 typically includes a modem 2272 or other means for establishing communications over the WAN 2273, such as the Internet. In a networked environment, program modules may be stored in a remote memory storage device. FIG. 21 illustrates, for example, that remote application programs 2285 can reside on remote computer 2280.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed In its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

An electrical property sensor is presented that includes a laminate structure including an insulating layer, a conductive layer and a conductive trace, the laminate structure having a first face separated from a second face by a thickness, the first face having a length and a width. The sensor also includes a first and second aperture, each of the first and second apertures extending from the first face of the laminate structure to the second face of the printed circuit board, the first and second aperture each include a receiving electrode and a transmitting electrode. When a fluid flows through the first aperture and an electric field is generated at the transmitting electrode, a sensor signal indicative of an electrical property is measured at the receiving electrode.

The sensor may be implemented such that the sensor signal is convertible to an electrical property value for the fluid, the electrical property value is a conductivity, an impedance or a dielectric constant.

The sensor may be implemented such that the first aperture is parallel to the length and perpendicular to the width.

The sensor may be implemented such that the first aperture has a first distance from an edge connector second aperture is parallel to the first aperture, and wherein a second center of the second aperture has a similar distance from the edge connector as a first center of the first aperture.

The sensor may be implemented such that the second aperture is parallel to the first aperture and the second aperture is at a second length from an edge connector, different from a first length of the first aperture from the edge connector.

The sensor may be implemented such that the first aperture has a first width, the second aperture has a second width, and the second width is greater than the first width.

The sensor may be implemented such that the second width is less than 20 mm.

The sensor may be implemented such that the second width is less than 1 mm.

The sensor may be implemented such that the second width is less than 0.5 mm.

The sensor may be implemented such that the second width is less than 300 μm.

The sensor may be implemented such that the first width is at least 50 μm.

The sensor may be implemented such that the first width is at least 100 μm.

The sensor may be implemented such that the fluid flows through the first aperture such that the fluid directly contacts the receiving electrode.

The sensor may be implemented such that the fluid flow is a first portion of a fluid flow and, when a second portion of the fluid flows through the second aperture, a second impedance signal is generated using the second transmitting and receiving electrodes.

The sensor may be implemented such that the second receiving electrode is decoupled from the first receiving electrode, such that the impedance signal and the second impedance signal differ.

The sensor may be implemented such that the sensor is part of a sensor stack composed of the impedance sensor and a second impedance sensor.

The sensor may also include a temperature sensor.

The sensor may be implemented such that the temperature sensor is electrically isolated from the fluid flow.

The sensor may be implemented such that the sensor includes a housing, and the housing is communicably coupled to an adapter for attachment to a dispensing system.

The sensor may be implemented such that a length of the laminate structure is more than twice the length of the first aperture.

The sensor may be implemented such that a length of the laminate structure is more than three times the length of the first aperture.

The sensor may be implemented such that a length of the laminate structure is more than four times the length of the first aperture.

The sensor may be implemented such that the laminate structure is a printed circuit board.

A sensing system for a mixture is presented that includes a sensing zone containing a mixture and a sensor within the sensing zone. The sensor includes a laminate structure including an insulating layer, a conductive layer and a conductive trace, a first and a second aperture within the laminate structure, each of the first and second apertures including a receiving electrode spaced apart from a transmitting electrode. The sensor is configured such that, while mixture is in direct contact with the transmitting electrode and the receiving electrode of each of the first and second apertures and when an electric field is generated at the transmitting electrode, a sensor signal is received at the receiving electrode of each of the first and second apertures. The system also includes a communication component that communicates a first electrical parameter for the mixture based on a first sensor signal from the receiving electrode of the first aperture, and a second electrical parameter based on a second sensor signal from the receiving electrode of the second aperture.

The system may be implemented such that the sensing zone is a container housing the mixture.

The system may be implemented such that the sensing system detects a difference between the first and second current signals and, based on the difference, indicates an instability in the mixture.

The system may be implemented such that the instability indicates sedimentation, creaming, entrained air, droplet formation or inconsistent mixing in the mixture.

The system may be implemented such that, based on the instability, a controller generates an inconsistency correction plan.

The system may be implemented such that the inconsistency correction plan includes mixing, de-gassing, or heating.

The system may be implemented such that the controller is configured to continue receiving current signals from the first and second aperture during the inconsistency correction plan.

The system may be implemented such that the sensing zone is a conduit through which the mixture flows.

The system may be implemented such that, to detect the instability, the controller is configured to, in situ, detect a difference between the first and second current signals, compare that difference to an acceptable threshold difference, and generate the instability indication if the difference exceeds the threshold difference.

The system may be implemented such that, based on a detection that the difference between the first and second current signals has decreased below the threshold difference, generating an indication that the instability is resolved.

The system may be implemented such that the sensing zone includes a mixing chamber that receives a first component flow and a second component flow.

The system may be implemented such that the sensing zone is within a dispenser configured to dispense the mixture.

The system may be implemented such that the electrical parameter is an impedance, a conductivity or a dielectric constant.

The system may be implemented such that the electrical parameter is indicative of a mixing ratio.

The system may be implemented such that the electrical parameter is indication of a fluid age.

The system may be implemented such that the electrical parameter is indicative of a cure progress.

The system may be implemented such that the transmitting electrode is perpendicular to a surface of the laminate structure.

The system may be implemented such that the transmitting electrode is aligned with a length of the aperture, and the receiving electrode is parallel to the transmitting electrode.

The system may be implemented such that the second aperture is parallel to the first aperture.

The system may be implemented such that a length of the laminate structure is more than twice the length of the first aperture.

The system may be implemented such that a length of the laminate structure is more than three times the length of the first aperture.

The system may be implemented such that a length of the laminate structure is more than four times the length of the first aperture.

The system may be implemented such that the first aperture has a first distance from an edge connector second aperture is parallel to the first aperture, and the second aperture has a similar distance from the edge connector.

The system may be implemented such that the second aperture is parallel to the first aperture and the second aperture is at a second length from an edge connector, different from a first length of the first aperture from the edge connector.

The system may be implemented such that the first aperture has a first width, the second aperture has a second width, and the second width is greater than the first width.

The system may be implemented such that the sensor is a first sensor, and further including a second sensor.

The system may be implemented such that the laminate structure is a first laminate structure, and the sensor includes: a second laminate structure, a third aperture within the second laminate structure including a third receiving electrode spaced apart from a third transmitting electrode, and the system is configured such that the fluid flows through the third aperture and directly contacts the third transmitting electrode and the third receiving electrode.

The system may be implemented such that the third aperture is positioned such that the fluid flows through the first aperture before flowing through the third aperture.

The system may be implemented such that the second laminate structure is coupled to the first laminate structure.

The system may be implemented such that the laminate structure includes a temperature sensor.

The sensor system may further includes: a housing that receives the sensor at an angle with respect to the fluid channel.

The system may be implemented such that the angle is less than 90°.

The system may be implemented such that the angle is less than 75°.

The system may be implemented such that the angle is less than 60°.

The system may be implemented such that the angle is less than 45°.

The system may be implemented such that the angle is less than 30°.

The system may be implemented such that the laminate structure is a printed circuit board.

A dispensing system is presented that includes a mixing unit configured to receives a first fluid stream and a second fluid stream and produces a mixture, a sensor within a fluid flow stream of the dispensing system. The sensor includes a laminate structure including an insulating layer, a conductive layer, and a conductive trace, a first aperture and a second aperture, each of the first and second apertures extending from a first face of the laminate structure to a second face of the laminate structure. Each of the first and second apertures includes: a transmitting electrode on a first portion of the aperture, a receiving electrode on a second portion of the aperture. The sensor is configured to receive a portion of the mixture through each of the first and second apertures, contacting both transmitting electrode and receiving electrode, and the sensor generates a sensor signal indicative of the mixture. The system also includes a dispenser configured to dispense the mixture, and a communication component configured to communicates the sensor signal.

The system may be implemented such that the sensor is downstream of the mixing unit.

The system may be implemented such that the sensor is upstream of the mixing and downstream from a first fluid source.

The system may be implemented such that the sensor is configured to directly contact the first fluid stream.

The system may be implemented such that the laminate structure is positioned within the fluid flow stream such that, during operation, the fluid flows through the aperture.

The system may be implemented such that the laminate structure is perpendicular to the fluid flow.

The system may be implemented such that the sensor is a first sensor, positioned downstream of the mixer, and the dispensing system includes a second sensor, positioned upstream of the mixer.

The system may be implemented such that the fluid flow includes a first component fluid flow and a second component fluid flow, and the sensor is configured to receive both the first component fluid flow and the second component fluid flow, simultaneously.

The system may be implemented such that the sensor is configured such that the first fluid flows through the aperture, the second fluid flows through a second aperture of the printed circuit board, the second aperture includes a second transmitting electrode and a second receiving electrode.

The system may include a housing that houses the sensor and physically separates the first fluid flow from the second fluid flow.

The system may be implemented such that the second sensor is placed in a first fluid stream, and further including a third sensor, placed in a second fluid stream upstream of the mixer.

The system may be implemented such that the laminate structure includes a third aperture including a third transmitting electrode and a third receiving electrode.

The system may be implemented such that, for each of the first and second electrodes, the transmitting electrode is parallel to a length of the aperture, and parallel to the receiving electrode.

The system may include an analyzer configured to receive the sensor signal and provides an indication.

The system may be implemented such that the indication includes an age of the first fluid.

The system may be implemented such that the analyzer is configured to determine the indication by comparing the sensor signal to a stored sensor signal.

The system may be implemented such that the indication includes a cure progress indication of the mixture.

The system may be implemented such that the indication includes a mix ratio.

The system may include including an analyzer configured to receive a first sensor signal from the first sensor, a second sensor signal from the second sensor, and a third sensor signal from the third sensor.

The system may be implemented such that the analyzer is configured to provide a mix ratio indication based on the received sensor signals.

The system may be implemented such that the analyzer is configured to provide a batch quality indication based on the received sensor signals.

The system may be implemented such that the analyzer is configured to provide an age indication based on the received sensor signals.

The system may be implemented such that the indication includes a mix quality across a cross section of the fluid flow.

The system may be implemented such that the analyzer is configured to determine the indication by applying a predictive model to the sensor signal.

The system may be implemented such that the indication includes an air bubble indication.

The system may be implemented such that the air bubble indication includes an indication that a sensed conductivity has spiked.

The system may be implemented such that the conductivity spike is greater than a relative threshold.

The system may be implemented such that, based on the indication, a control signal is generated to purge the fluid flow.

The system may be implemented such that, in response to the sensor signal, a control signal is provided to a motor to adjust a motor speed of the motor.

The system may be implemented such that, in response to the sensor signal, a purge is automatically initiated.

The system may include a display component that receives the sensed signal and provides a visual indication of the sensed signal.

The system may be implemented such that the visual indication is a mix quality indication.

The system may be implemented such that the communication component provides the sensed signal to a datastore.

The system may be implemented such that the sensor includes a temperature sensor.

The system may be implemented such that the sensor is coplanar with the receiving and transmitting electrodes.

The system may be implemented such that the temperature sensor is isolated from the fluid flow.

The system may be implemented such that the temperature sensor is isolated by a layer of varnish or a layer of epoxy-resin based adhesive.

The system may be implemented such that the sensor is a first sensor, and further including a second sensor coupled to the first sensor.

The system may be implemented such that the coupling includes a conductive material.

The system may be implemented such that the conductive material is solder.

The system may be implemented such that the second sensor is coupled such that the fluid flows through the first aperture before flowing through a second aperture, the second sensor includes the second aperture.

The system may be implemented such that the first sensor is a four-layer laminate structure and the second sensor is a two-layer laminate structure.

The system may be implemented such that the laminate structure is non-orthogonally angled with respect to the fluid flow.

The system include a pressure sensor that detects a pressure indication at an outlet of a reservoir or pump.

The system may be implemented such that communication component communicates the pressure signal substantially in real-time.

The system may include a pump system that continuously pumps fluid between a dispenser inlet and a reservoir.

The system may include an analyzer that receives the signal indicative of the mixture and provides a mixture indication to a device.

The system may be implemented such that the analyzer, based on the signal indicator of the mixture, generates the mixture indication.

The system may be implemented such that the analyzer converts the signal indicative of the mixture into the mixture indication.

The system may include an analyzer that receives the pressure signal and provides a pressure indication to a device.

The system may be implemented such that the analyzer, based on the pressure signal, generates the pressure indication.

The system may be implemented such that the analyzer converts the pressure signal into the pressure indication.

The system may be implemented such that the dispensing system includes a sensor housing that houses the sensor.

The system may be implemented such that the sensor housing maintains the sensor at an angle with respect to the fluid flow.

The system may be implemented such that the angle is less than 75°.

The system may be implemented such that the angle is less than 60°.

The system may be implemented such that the angle is less than 45°.

The system may be implemented such that the angle is less than 30°.

The system may be implemented such that the sensor housing and the dispenser form a unitary body.

The sensor may be implemented such that the sensor housing couples to an adapter that communicably couples fluid flow from the dispenser through the sensor housing.

The sensor may be implemented such that the laminate structure is a printed circuit board.

A signal analysis system for a plurality of dispensing systems is presented that includes a first dispensing system with a first sensor that detects a first sensor signal from a first sensor within the first dispensing system, the first sensor including a first laminate structure, a second dispensing system with a second sensor that detects a second sensor signal from a second sensor within the first dispensing system, the second sensor including a second laminate structure. The system includes an analyzer that receives the first and second sensor signals and generates a first sensor output and a second sensor output. The signal analysis system provides the first sensor output to a first device, and the second sensor output to a second device.

The system may be implemented such that the first sensor signal is received and first output provided in situ.

The system may be implemented such that the sensor signal is received using a wireless protocol.

The system may be implemented such that the output is communicated to the first device using a wireless protocol.

The system may be implemented such that the first sensor includes a transmitting electrode and a receiving electrode, each electrode being coplanar with a surface of the laminate structure.

The system may be implemented such that the set of electrodes each have a width.

The system may be implemented such that one of the electrodes on the first sensor has a first width, a second electrode has a second width, and the second width is wider than the first width.

The system may be implemented such that the set of electrodes on the first sensor each have a first end and a second end, and each of the electrodes has a distance measured from an edge connector to the first end.

The system may be implemented such that the set of electrodes on the first sensor includes a first electrode with a first electrode first end and a first electrode second end, a second electrode with a second electrode first end and a second electrode second end. A first distance, measured from the first electrode first end and an edge connector, and a second distance, measured from the second electrode first end, and the first distance and the second distance are different.

The system may be implemented such that each of the set of electrodes on the first sensor have a width and a length.

The system may be implemented such that the set of electrodes on the first sensor are arranged linearly, along a length of the board.

The system may be implemented such that the set of electrodes on the first sensor are arranged linearly, along a width of the board.

The system may be implemented such that the first sensor output is a subset of data obtained the first sensor signal, and the second device requested the subset of data.

The system may be implemented such that the system includes an MQTT broker that facilitates communication of the first sensor output based on the request of the second device.

The system may be implemented such that the first laminate structure is a printed circuit board.

An electrical property sensor is presented that includes a laminate structure including an insulated layer, a conductive layer and a conductive trace, the laminate structure having a first face separated from a second face by a thickness, the first face having a length and a width. The sensor includes a first sensing area configured to sense a first electrical parameter value. The sensor includes a second sensing area configured to sense a second electrical parameter value, the second sensing area is decoupled from the first sensing area. When a fluid directly contacts the first sensing area, when actuated, generates an electric field, such that a signal is measured at a receiving electrode.

The sensor may be implemented such that the first sensing area and the second sensing area are coplanar.

The sensor may be implemented such that the laminate structure includes a flexible substrate.

The sensor may be implemented such that the flexible substrate is configured to operate in a first configuration, where the first sensing area and the second sensing area are coplanar, and in a second configuration, where the first sensing area is parallel to the second sensing area, separated by a gap.

The sensor may be implemented such that the laminate structure is a first laminate structure, the first laminate structure includes the first sensing area. A second laminate structure includes the second sensing area.

The sensor may be implemented such that the first sensing area is configured to sense the first electrical parameter value using surface sensing techniques.

The sensor may be implemented such that the first sensing area is configured for placement in-line with a flow direction of the fluid.

The sensor may be implemented such that the laminate structure includes an aperture, and the first sensing area includes a surface angled with respect to the first face and the second face, such that the sensor is configured to sense the electrical parameter value as a flow of fluid passes through the aperture.

The sensor may be implemented such that one of the first and second sensing areas is configured to serve as either the transmitting electrode, a receiving electrode or a ground electrode.

The sensor may be implemented such that the laminate structure includes a third and a fourth sensing area.

The sensor may be implemented such that the first, second, third and fourth sensing areas are coplanar with each other.

The sensor may be implemented such that the laminate structure includes a flexible substrate, and the first, second, third and fourth sensing areas are curved.

The sensor may be implemented such that a conduit includes the first, second, third and fourth sensing areas.

The sensor may be implemented such that the laminate structure includes a third and a fourth sensing area, and the first, second, third and fourth sensing areas are configured to operate as receiving electrodes. A conduit includes the first, second, third and fourth sensing areas. A transmitting electrode is spaced apart from each of the first, second, third and fourth sensing areas.

The sensor may be implemented such that the receiving electrode and the transmitting electrodes are arranged in an interdigitated comb on the first surface.

The sensor may be implemented such that the electrical property value includes a conductivity, an impedance, or a dielectric constant.

The sensor may be implemented such that the fluid is an adhesive.

The sensor may be implemented such that the first sensing area includes a first aperture in the PCB, the second sensing area includes a second aperture in the laminate structure, and the sensor includes a connection end and the first aperture is a first distance from the connection edge, and the second sensing area includes a second distance from the connection end, and the first and second distances are different.

The sensor may be implemented such that the connection end includes an edge connector and the first aperture, second aperture and the edge connector are collinear.

The sensor may be implemented such that the sensor includes a housing that receives the laminate structure.

The sensor may be implemented such that the laminate structure is a printed circuit board.

An electrical property sensing system is presented that includes a sensor configured to directly contact a fluid. The sensor includes a transmitting electrode configured to, when actuated generate an electric field, a receiving electrode configured to generate an electrical parameter signal for the fluid. The system includes a communication component configured to: receive the electrical parameter signal, generate a signal analysis based on the electrical parameter signal, and generate a feedback signal based on the electrical parameter signal, the feedback signal is generated in real-time.

The sensing system may include a housing configured to receive the sensor, the housing is configured to interact with a sensing zone.

The sensing system may be implemented such that the sensing zone includes a static fluid.

The sensing system may be implemented such that the sensing zone receives a flow of the fluid.

The sensing system may be implemented such that the sensor is a first sensor, and the sensing system includes a second sensor.

The sensing system may be implemented such that the sensing system includes a laminate structure including an insulated layer, a conductive layer and a conductive trace, and the laminate structure includes the first and second sensor.

The sensing system may be implemented such that the first and second sensor are coplanar with respect to the laminate structure.

The sensing system may be implemented such that the laminate structure is flexible.

The sensor may be implemented such that the sensing system includes a first laminate structure, and a second laminate structure, and the first laminate structure includes the transmitting electrode and the second laminate structure includes the receiving electrode.

The sensing system may be implemented such that the receiving electrode is a first receiving electrode, and the sensing system includes a second receiving electrode.

The sensing system may be implemented such that the sensing system first receiving electrode, the second receiving electrode, and the transmitting electrode form a portion of a conduit.

The sensing system may be implemented such that the fluid flows through the conduit.

The sensing system may be implemented such that the feedback signal includes a mix ratio, a purge signal, a detected air bubble or an unstable mixture.

The sensing system may be implemented such that the electrical parameter is a conductivity, an impedance, or a relative permittivity.

The sensing system may be implemented such that the sensing system is incorporated into a dispenser, the dispenser having a pressure sensor, and the sensing system further includes processing circuitry and a memory configured to: receive a pressure signal, receive the electrical parameter value, determine, based on the pressure signal and the electrical parameter value, a setting change needed for the dispenser.

The sensing system may be implemented such that the communication component communicates the setting change to a user of the dispenser.

The sensing system may be implemented such that the communication component generates a command to change the setting.

The sensing system may be implemented such that the laminate structure includes a printed circuit board.

An electrical parameter sensing system is presented that includes a laminate structure including an insulated layer, a conductive layer and a conductive trace, the laminate structure including a plurality of sensing areas, the sensing areas configured to directly contact a fluid, an edge connector electrically coupled to each of the plurality of sensing areas, a signal reader configured to receive a sensed electrical parameter from the edge connector, and the signal reader is configured to change a configuration of the plurality of sensing areas from a first configuration to a second configuration. The first configuration includes a first sensing area as a transmitting electrode or ground electrode and a second sensing area as a receiving electrode, and the second configuration includes the first sensing area as a receiving electrode and the second sensing area as a transmitting electrode or ground. The system includes a communication component configured to communicate a sensed electrical parameter value, the parameter value is sensed by the receiving electrode when the transmitting electrode generates an electric field.

The sensing system may include a housing with a conduit for fluid flow, and the sensing areas form a portion of the housing.

The sensing system may be implemented such that the sensing areas are in line with a direction of the fluid flow.

The sensing system may be implemented such that the signal reader includes a multiplexor that changes the configuration of the plurality of sensing areas periodically.

The sensing system may include a signal analyzer that, based on a signal the receiving electrode generates a feedback signal.

The sensing system may be implemented such that the feedback signal includes a mix ratio, a purge signal, or a detected inconsistency.

The sensing system may be implemented such that the feedback signal includes a correction indication.

The sensing system may be implemented such that the signal reader includes the signal analyzer.

The sensing system may be implemented such that the sensing areas sense the electrical parameter using surface sensing techniques.

The sensing system may be implemented such that a device includes the signal reader, the signal analyzer and a display configured to display the sensed electrical parameter value.

The sensing system may be implemented such that the sensing areas sense the electrical parameter using bulk sensing techniques.

The sensing system may be implemented such that the laminate structure is flexible.

The sensing system may be implemented such that the laminate structure includes a molded material.

The sensing system may be implemented such that the laminate structure is a printed circuit board.

A method of detecting an inconsistency in a fluid is presented that includes receiving a sensed electrical parameter, using a signal reader, from a sensor, the sensor is in direct contact with the fluid. The sensor includes a PCB, the PCB having a transmitting electrode and a receiving electrode, the electrical parameter is sensed by the receiving electrode when the transmitting electrode is actuated. The method includes detecting, using a signal analyzer, based on the sensed electrical parameter, an inconsistency in the fluid, generating a correction indication for the inconsistency, and communicating the correction indication, using a communication component.

The method may be implemented such that communicating includes communicating the correction indication to a device with a display, such that the correction indication is presented on the display.

The method may be implemented such that the device includes the signal reader and the signal analyzer.

The method may be implemented such that the device includes a multiplexer.

The method may be implemented such that the sensor includes a second receiving electrode, the transmitting electrode, receiving electrode and the second receiving electrode are electrically coupled to an edge connector, and the signal reader receives the edge connector.

The method may be implemented such that the second receiving electrode, the transmitting electrode and the receiving electrode are in line with a flow of fluid, such that the flow of fluid contacts the surface of the laminate structure during flow.

The method may be implemented such that the steps of receiving, detecting and generating are done in real-time.

The method may be implemented such that the inconsistency includes: an amount of cure, a mix ratio, entrained air, or mix instability.

The method may be implemented such that the correction indication includes: a dispensing parameter change or a purge indication.

The method may be implemented such that the correction indication includes a command that causes a dispenser to automatically implement the correction indication.

The method may be implemented such that the sensor senses the electrical parameter value using bulk sensing techniques.

The method may be implemented such that the sensor senses the electrical parameter using surface sensing techniques.

The method may be implemented such that the laminate structure is flexible.

The method may be implemented such that the laminate structure includes more receiving electrodes than transmitting electrodes.

The method may be implemented such that the laminate structure includes an aperture and the aperture includes the transmitting and receiving electrodes.

The method may be implemented such that the sensed electrical parameter includes an impedance, a conductivity, or a dielectric constant.

The method may be implemented such that the fluid is an adhesive.

The method may be implemented such that the laminate structure is a printed circuit board.

A fluid parameter sensor is presented that includes a flexible laminate structure including an electrode pair, the electrode pair including a transmitting electrode and a receiving electrode. An indication is sensed at the receiving electrode when the fluid is in direct contact the receiving electrode, and when the transmitting electrode is actuated. The sensor includes a signal analyzer that, based on the indication, generates a fluid parameter value and a communication component configured to communicate the sensed fluid parameter value.

The fluid parameter sensor may be implemented such that the electrode pair is a first electrode pair, and the flexible laminate structure includes a second electrode pair, decoupled from the first electrode pair.

The fluid parameter sensor may be implemented such that the second electrode pair includes the transmitting electrode and a second receiving electrode, different from the receiving electrode.

The fluid parameter sensor may be implemented such that the flexible laminate structure includes a third electrode pair including the transmitting electrode and a third receiving electrode.

The fluid parameter sensor may be implemented such that the second electrode pair includes a second transmitting electrode and a second receiving electrode.

The fluid parameter sensor may be implemented such that the transmitting electrode and receiving electrode are printed onto the laminate structure, such that direct contact includes the fluid contacting a surface of the laminate structure.

The fluid parameter sensor may be implemented such that the transmitting electrode is printed on a first portion of the laminate structure, the receiving electrode is printed on a second portion of the laminate structure such that, and the laminate structure is configured to receive a flow of fluid between the transmitting electrode and the receiving electrode.

The fluid parameter sensor may be implemented such that the laminate structure is configured to fold from a flat configuration to the configuration configured to receive the fluid flow.

The fluid parameter sensor may include an edge connector.

The fluid parameter sensor may be implemented such that a signal receiver receives the edge connector.

The fluid parameter sensor may include a near-field communication tag.

The fluid parameter sensor may include a radio-frequency identification tag.

The fluid parameter sensor may be implemented such that the transmitting and receiving electrodes are arranged in an interdigitated comb structure.

The fluid parameter sensor may be implemented such that the receiving electrode senses the indication by surface sensing techniques.

The fluid parameter sensor may be implemented such that the receiving electrode senses the indication by bulk sensing techniques.

The fluid parameter sensor may be implemented such that the laminate structure is a printed circuit board.

### EXAMPLES

These examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed considering the number of reported significant digits and by applying ordinary rounding.

### Example 1: Sensor Application for Mix Ratio Monitoring for Paint Spray

A system consisting of a paint mixing cup (3M™ PPS™ Series 2.0 Spray Cup, available from 3M Company, St. Paul, MN) was used to contain a hand-mixed automotive clear coat paint formulation (Standocryl VOC Xtra Clear K9560, include Clear K9560, Thinner 5820, and Hardener VOC 25-30, available from Standox GmbH, Wuppertal, Germany). A PPS™ liner was filled with the base mixture, only Clear K9250 without Thinner and Hardener, and tested with a sensor (as illustrated in FIG. 9) that was embedded into a stir stick that was moved through the mixture. The stir stick was configured to retrieve and provide in-situ conductivity and dielectric constant signals to a connector box where a software program took the signals and created data that was graphed over time for conductivity and dielectric constant measurements.

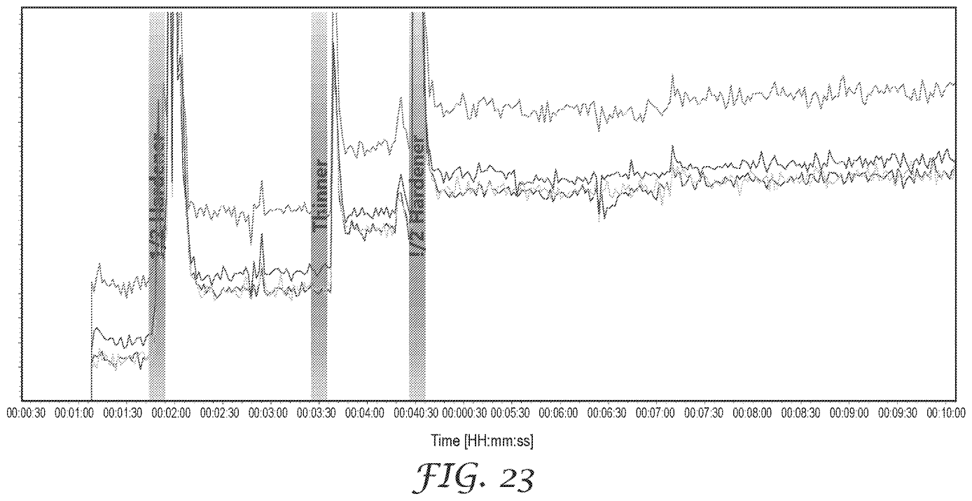

Using the system described above, a set of experiments were conducted which varied the mix ratio of three components (Clear K9560, Thinner 5820, and Hardener VOC 25-30) to form the paint mixture. The formulation of the clear Standocryl VOC Xtra Clear K9560 is, 70.42 % by weight of Clear K9560, 25.35 % by weight of Thinner 5820, and 4.23 % by weight of Hardener VOC 25-30. First half of the Hardener VOC 25-30 was added to the base mixture, only Clear K9560 (see before), and mixed. In real-time, conductivity and relative permittivity signals were collected, recorded, and were plotted vs. time and shown in FIG. 22 and FIG. 23, respectively. An instant drop in solution conductivity was observed while the dielectric constant increased. Next, an addition of Thinner 5820 was made to the mixture. A significant increase in mixture conductivity and dielectric constant were observed. A final addition of the second half of the Hardener VOC 25-30 was made to the mixture. Conductivity dropped again and the dielectric constant increased.

### Example 2

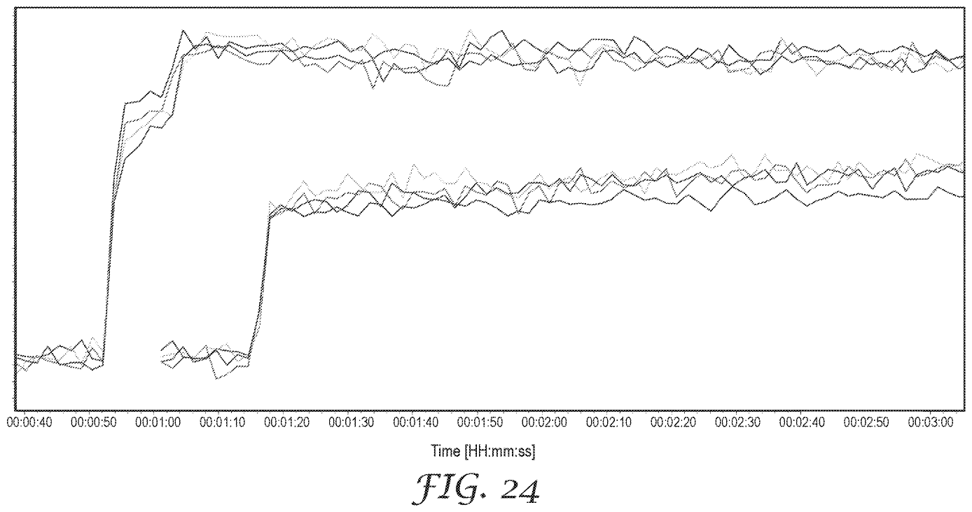

A system consisting of a container was used to hold a liquid material that is low viscosity in one state and high viscosity in another state. This material would be subjected to mixing from a sensor embedded stir stick (as illustrated in FIG. 9). The material would be low viscosity in one state and high viscosity in another state. The stir stick was configured as in FIG. 9 and was allowed to retrieve conductivity signals through a reader to a connector box where a software program took the signals and created data that was graphed over time for conductivity measurements. The initial measurement readings in the chart would correlate to those for measuring air while once the sensor stick was introduced to the material, a spike and increase in conductivity was observed for each material state over time. The retrieved data is illustrated in FIG. 24.

1. An electrical property sensor comprising:
a laminate structure comprising an insulating layer, a conductive layer and a conductive trace, the laminate structure having a first face separated from a second face by a thickness, the first face having a length and a width;
a first and second aperture, each of the first and second apertures extending from the first face of the laminate structure to the second face of the printed circuit board, the first and second aperture each comprise a receiving electrode and a transmitting electrode; and
wherein, when a fluid flows through the first aperture and an electric field is generated at the transmitting electrode, a sensor signal indicative of an electrical property is measured at the receiving electrode.

2. The sensor of claim 1, wherein the sensor signal is convertible to an electrical property value for the fluid, wherein the electrical property value is a conductivity, an impedance or a dielectric constant.

3. The sensor of claim 1, wherein the first aperture is parallel to the length and perpendicular to the width.

4. The sensor of claim 3, wherein the first aperture has a first distance from an edge connector second aperture is parallel to the first aperture, and wherein a second center of the second aperture has a similar distance from the edge connector as a first center of the first aperture.

5. The sensor of claim 4, wherein the second aperture is parallel to the first aperture and wherein the second aperture is at a second length from an edge connector, different from a first length of the first aperture from the edge connector.

6. The sensor of claim 1, wherein the first aperture has a first width, the second aperture has a second width, and wherein the second width is greater than the first width.

7. The sensor of claim 1, wherein the fluid flows through the first aperture such that the fluid directly contacts the receiving electrode.

8. The sensor of claim 1, wherein the fluid flow is a first portion of a fluid flow and, when a second portion of the fluid flows through the second aperture, a second impedance signal is generated using the second transmitting and receiving electrodes.

9. The sensor of claim 1, and further comprising a temperature sensor.

10. The sensor of claim 1, wherein the sensor comprises a housing, and wherein the housing is communicably coupled to an adapter for attachment to a dispensing system.

11. The sensor of claim 1, wherein a length of the laminate structure is more than twice the length of the first aperture.

12-13. (canceled)

14. The sensor of claim 1, wherein the laminate structure is a printed circuit board.

15. A signal analysis system for a plurality of dispensing systems, the system comprising:
a first dispensing system with a first sensor that detects a first sensor signal from a first sensor within the first dispensing system, the first sensor comprising a first laminate structure;
a second dispensing system with a second sensor that detects a second sensor signal from a second sensor within the first dispensing system, the second sensor comprising a second laminate structure;
an analyzer that receives the first and second sensor signals and generates a first sensor output and a second sensor output; and
wherein the signal analysis system provides the first sensor output to a first device, and the second sensor output to a second device.

16. The system of claim 15, wherein the first sensor signal is received and first output provided in situ.

17. (canceled)

18. The system of claim 15, wherein the first sensor comprises a transmitting electrode and a receiving electrode, each electrode being coplanar with a surface of the laminate structure.

19. The system of claim 15, wherein one of the electrodes on the first sensor has a first width, a second electrode has a second width, and wherein the second width is wider than the first width.

20. The system of claim 19, wherein the set of electrodes on the first sensor each have a first end and a second end, and each of the electrodes has a distance measured from an edge connector to the first end wherein the set of electrodes on the first sensor comprises a first electrode with a first electrode first end and a first electrode second end, a second electrode with a second electrode first end and a second electrode second end, and wherein a first distance, measured from the first electrode first end and an edge connector, and a second distance, measured from the second electrode first end, and wherein the first distance and the second distance are different.

21. The system of claim 20, wherein the set of electrodes on the first sensor are arranged linearly, along a length or width of the board.

22. (canceled)

23. The system of claim 20, wherein the first sensor output is a subset of data obtained the first sensor signal, and wherein the second device requested the subset of data.

24-25. (canceled)

26. A method of detecting an inconsistency in a fluid, the method comprising:
receiving a sensed electrical parameter, using a signal reader, from a sensor, wherein the sensor is in direct contact with the fluid;
wherein the sensor comprises a PCB, the PCB having a transmitting electrode and a receiving electrode, wherein the electrical parameter is sensed by the receiving electrode when the transmitting electrode is actuated;
detecting, using a signal analyzer, based on the sensed electrical parameter, an inconsistency in the fluid;
generating a correction indication for the inconsistency; and
communicating the correction indication, using a communication component.

27-40. (canceled)
